(19) United States
(12) Patent Application Publication
(10) Pub. No.: US 2023/0414784 A1
(43) Pub. Date: Dec. 28, 2023

(54) Cationic Bottlebrush Polymers for Gene Delivery

(71) Applicant: Regents of the University of Minnesota, Minneapolis, MN (US)

(72) Inventors: Theresa REINEKE, Vadnais Heights, MN (US); Rishad DALAL, Minneapolis, MN (US)

(21) Appl. No.: 18/340,592

(22) Filed: Jun. 23, 2023

Related U.S. Application Data
(60) Provisional application No. 63/366,985, filed on Jun. 24, 2022.

(51) Int. Cl. A61K 48/00; C08F 293/00; A61K 47/58

(52) U.S. Cl. CPC A61K 48/0041; C08F 293/005; A61K 47/58; C08F 2438/03

(57) ABSTRACT

An example compound includes a bottlebrush polymer associated with a biological agent. The bottlebrush polymer may include a backbone and a plurality of side-chains covalently bonded to the backbone. Each side-chain of the plurality of side-chains may include a plurality of repeating cationic units.

This application claims the benefit of U.S. Provisional Patent Application No. 63/366,985, filed Jun. 24, 2022, the entire contents of which is incorporated herein by reference.

### GOVERNMENT INTEREST

This invention was made with government support under Grant No. N660011824041 awarded the Department of Defense/Defense Advanced Research Projects Agency (DARPA). The government has certain rights in the invention.

### BACKGROUND

Gene delivery is the process of introducing a biological agent (e.g., a nucleic acid) into host cells. Typically, the biological agent is part of a vector, which is a vehicle designed to carry the biological payload into another cell. Vectors may be broadly classified into two categories, viral and non-viral. Engineered viral vectors are commonly used in gene therapy, but viral vectors present many challenges, especially at a scale suitable for large patient populations. Synthetic polymers as non-viral vectors for gene therapy are an attractive option to replace viral vectors.

### SUMMARY

Cationic polymer vehicles are promising platforms for nucleic acid delivery because of their scalability, biocompatibility, and chemical versatility. Advancements in synthetic polymer chemistry allow for precisely tuned chemical functionality with various macromolecular architectures to increase the efficacy of nonviral-based gene delivery. In general, the present disclosure is directed to polymer architectures that increase the efficacy of biomacromolecular payload such as, for example, pDNA, RNP, and the like, relative to linear polymers, which may be used as building blocks of polymer architectures according to the present disclosure.

The disclosure is generally directed to cationic bottlebrush polymers. Bottlebrush polymers of the present disclosure may be synthetically defined unimolecular structures which increase the efficacy of nonviral-based gene delivery. In some examples a bottlebrush polymer may include a backbone and a plurality of side-chains covalently bonded to the backbone. In some examples, each side chain of the plurality of side-chains may include a repeating cationic unit. The repeating cation unit synthesized in the form of a bottlebrush polymer may be far more efficient in functional biological agent efficiency (e.g., pDNA) than the linear analogue of the repeating cationic unit. Accordingly, architectural modification of polymer-based delivery vehicles through the creation of bottlebrush polymers may be advantageous for delivery of biomacromolecules. In some examples, bottlebrush polymers according to the present disclosure may yield up to a 60-fold increase in % EGFP+ cells in comparison to a linear macromonomer. Additionally, quantitative confocal analysis revealed that bottlebrushes were able to shuttle plasmid DNA (pDNA) into and around the nucleus more successfully than pDNA delivered via linear analogues.

In some examples, the disclosure is directed to a compound including a bottlebrush unimolecular polymer. The bottlebrush unimolecular polymer includes a backbone and a plurality of side-chains covalently bonded to the backbone. Each side-chain of the plurality of side-chains includes a plurality of repeating cationic units.

In some examples, the disclosure is directed to a compound including a bottlebrush unimolecular polymer. The bottlebrush unimolecular polymer includes a backbone and a plurality of side-chains covalently bonded to the backbone. Each side-chain of the plurality of side-chains includes a plurality of repeating cationic units. The compound further includes a biological agent associated with the bottlebrush unimolecular polymer.

In some examples, the disclosure is directed to a technique which includes synthesizing a bottlebrush unimolecular polymer including a backbone and a plurality of side-chains covalently bonded to the backbone. Each side-chain of the plurality of side-chains includes a repeating cationic unit. The technique includes associating the bottlebrush unimolecular polymer with a biomacromolecule to form a bottleplex.

In some examples, the disclosure is directed to a technique including selecting a volume of a composition. The composition includes cell a bottlebrush unimolecular polymer. The bottlebrush unimolecular polymer includes a backbone and a plurality of side-chains covalently bonded to the backbone. Each side-chain of the plurality of side-chains includes a repeating cationic unit. The composition also includes a biological payload associated with the bottlebrush unimolecular polymer, and a pharmaceutically acceptable liquid carrier. The technique further includes applying the volume of the composition to a cell.

In some examples, the disclosure is directed to a non-viral bottleplex comprising a bottlebrush unimolecular polymer and a biological agent associated with the bottlebrush polymer. The bottlebrush polymer includes a backbone and a plurality of side-chains covalently bonded to the backbone. Each side-chain of the plurality of side-chains includes a repeating cationic unit. The biological agent associated with the bottlebrush unimolecular polymer is chosen from pDNA, RNP, and mixtures and combinations thereof.

In some examples, the disclosure is directed to a technique which includes synthesizing a bottlebrush unimolecular polymer. Synthesizing a bottlebrush unimolecular polymer includes performing reversible addition-fragmentation chain transfer polymerization of a plurality of cationic monomers to create a plurality of macromonomers. Synthesizing a bottlebrush unimolecular polymer also includes performing ring-opening metathesis polymerization to covalently bond the plurality of macromonomers to form a backbone.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 illustrates the relatively higher delivery efficiency of an example bottlebrush polymer over an example linear building block of the bottlebrush polymer.

FIG. 2A illustrates an example chemical scheme showing a two-step orthogonal polymerizations of reversible addition-fragmentation (RAFT) polymerization followed by ring-opening metathesis polymerization (ROMP) to achieve bottlebrush polymers with pendant tertiary amines.

FIG. 2B is a conceptual illustration of example bottlebrush (BB) polymers according to the present disclosure synthesized from one macromonomer (MM).

FIG. 3 illustrates Stacked H NMR spectra showing the conversion of peak a to a′ in an example macromonomer according to the present disclosure (top) and a bottlebrush polymer according to the present disclosure (bottom).

FIG. 4 illustrates an example stacked normalized differential refractive index (RI) showing conversion of a macromonomer according to the present disclosure to example bottlebrush polymers.

FIG. 5 illustrates nomenclature of the present disclosure, showing nomenclature of degree of polymerization of side-chain (N_{sc}) and the backbone (N_{bb}).

FIG. 6 illustrates standardized protonation states of an example macromonomer and four example bottlebrush polymers calculated from titration data. Dotted line shows percent of polymer protonated at pH 7.4.

FIG. 7 illustrates fluorescence analyzed through dye exclusion of PicoGreen of an example macromonomer and four example bottlebrush polymers according to the present disclosure.

FIG. 8 illustrates Rb data acquired from DLS of an example polyplex formed from an example macromonomer and a biological agent and example bottleplexes formed from example bottlebrush polymers with a biological agent, formed in water (t=0 min) and then aggregation of the complexes over 60 min after addition of Opti-MEM. Error bars are represented by ±standard deviation of analyzed data (n=3).

FIG. 9 illustrates flow cytometry data showing percent EGFP positive HEK293 cells due to delivery with an example macromonomer and four example bottlebrush polymers according to the present disclosure, at N/P ratios of 5, 7.5, and 10. The asterisks (*) indicate statistical difference (p<0.05) analyzed by one-way ANOVA followed by a post hoc Tukey test.

FIG. 10 illustrates cell viability data as measured via CCK-8 assay with HEK293 cells with complexes formed at N/P ratios of 5, 7.5, and 10. Error bars are represented by ±standard deviation of analyzed data (n=3).

FIG. 11 illustrates confocal 2D images comparing example macromonomer (MM) and example bottlebrush polymer (BB_37) by tracking Cy5-labeled pDNA internalized into HEK293 cells, cells fixed at 24 h. Confocal microscopy observations: nuclear Hoechst stain (left), lysosomal LAMP-2 stain (second from left), Cy5-pDNA fluorescent tag (third from left), and EGFP produced (second from right).

FIG. 12 illustrates Confocal 3D images comparing MM and BB_37 by tracking Cy5 labeled pDNA internalized into EGFP positive cells. Confocal microscopy observations: nuclear, EGFP in cell cytoplasm, Cy5-pDNA in nucleus, and Cy5-pDNA in cytoplasm.

FIG. 13 illustrates normalized volume (μm³) of pDNA per EGFP positive cells in the nucleus and cytoplasm when transfected with example macromonomer MM and example bottlebrush BB_37, determined by quantitative image analysis 24 h after initial transfection.

FIG. 14 illustrates the distribution of polyplexes/bottleplexes from the periphery of the nucleus in EGFP+ and EGFP− cells analyzed from confocal analysis. Negative distance lengths indicate the centroid of the pDNA complex is within the nucleus.

FIG. 15 illustrates an H NMR stack of example macromonomer MM and four bottlebrush polymers showing a decrease in alkene ring closed norborene peak at a ppm shift with the inset box. The illustrated NMRs were taken in heavy water.

FIG. 16 illustrates the differential refractive index for different concentrations of example macromonomer MM.

FIG. 17 illustrates the differential refractive index for different concentrations of example bottlebrush polymer BB_13.

FIG. 18 illustrates titration data of the example macromonomer and four example bottlebrush polymers according to the present disclosure.

FIG. 19 illustrates dynamic light scattering (DLS) data of the % intensity of the example polyplex and the four examples bottleplexes formed in water.

FIG. 20 illustrates dynamic light scattering (DLS) data of the % number average of the example polyplex and the four examples bottleplexes formed in water.

FIG. 21 illustrates gel electrophoresis of the polyplex formed the example macromonomer and associated biological agent and four example bottleplexes formed from the example bottlebrush polymers and a respective biological agent, showing complete binding at an N/P ratio of 1.5 and greater.

FIG. 22 illustrates dye exclusion data showing the amount of fluorescence of PicoGreen before and after the addition of 10% FBS solution.

FIG. 23 illustrates the zeta potential of pDNA, MM polyplex, and four example bottleplexes at an N/P ratio of 7.5.

FIG. 24 illustrates the percent EGFP-positive (EGFP+) cells transfected with example macromonomer MM and example bottlebrushes BB_13, BB_20, BB_26, and BB_37 at N/P ratios of 7.5, 10, and 12.5.

FIG. 25 illustrates the gating scheme used for identifying the amount of EGFP+ cells. Three representative examples are shown in pDNA, MM at N/P 7.5, and BB_37 at N/P 7.5.

FIG. 26 illustrates the viability of cells transfected with example macromonomer MM, and example bottlebrushes BB_13, BB_20, BB_26, and BB_37 at N/P ratios of 5, 7.5, and 10.

FIG. 27 illustrates the percent Cy5 and EGFP+ cells at N/P ratios of 2.5 and 7.5, with EGFP illustrated in solid and Cy5 illustrated as dashed.

FIG. 28 illustrates the percent Cy5 and EGFP cells and geometric MFI of Cy5 transfected with MM, BB_13, BB_20, BB_26, and BB_37 at N/P ratios of 7.5 after 24 hours elapsed.

FIG. 29 illustrates the gating scheme used for identifying the amount of Cy5-positive (Cy5+) cells. Three representative examples are shown in pDNA, MM at N/P of 7.5, and BB_37 at N/P of 7.5.

FIG. 30 illustrates Microscope images of HEK293 cells 24 h after initial transfection with a GFP filter to image EGFP positive cells.

FIG. 31 illustrates confocal 2D images comparing MM and BB_37 by tracking Cy5 labeled pDNA internalized into HEK293 cells, cells fixed at 24 h.

FIG. 32 illustrates colocalization of Lamp-2-stained lysosome and Cy5 tagged pDNA.

FIGS. 33-35 are flowcharts illustrating example techniques in accordance with one or more examples of the present disclosure.

FIG. 36 is a flowchart illustrating an example technique for synthesizing a bottlebrush unimolecular polymer in accordance with one or more aspects of the present disclosure.

### DETAILED DESCRIPTION

Despite the vast curative potential of gene therapy, widespread clinical deployment faces an uncertain outlook due to excessive reliance on engineered viral vectors, which can be used to deliver therapeutic biomacromolecule payloads such as, for example, messenger RNA (mRNA), plasmid DNA (pDNA), ribonucleoprotein (RNP) (e.g., CRISPR/Cas9 RNP), antisense oligonucleotides (ASOs), and small interfering RNA (siRNA). However, the high costs, lengthy delays and regulatory challenges involved in manufacturing clinical grade viruses at scale for large patient populations have imposed severe logistical bottlenecks. In addition to manufacturing and regulatory delays, the cargo capacity of viral vectors is limited, and this size ceiling is particularly problematic in the context of large biological payloads.

Although advances in virus manufacturing have minimized occurrences of carcinogenic mutations, genomic integration and fatal systemic inflammatory responses, these risks are amplified when repeated dosing or large dosages are involved. For many types of gene therapy (e.g., CRISPR therapeutics) to become safe, scalable, and affordable, there is a need to identify synthetic substitutes for viral carriers.

Polymeric gene delivery vehicles have been used in clinical gene therapy due to their versatility, relative low production cost, and low immunogenicity. Synthetic polymers can deliver biomacromolecule payloads such as, for example, pDNA, RNP, and the like, due to their versatility, low toxicity, and the ability to encapsulate large payloads. Some recent examples indicate that synthetic polymer-based systems achieved biomacromolecule based gene delivery and gene editing both in vitro and in vivo.

For example, in aqueous physiological solutions, cationic polymers can associate with (e.g., spontaneously bind to) negatively charged pDNA and form interpolyelectrolyte complexes. These complexes are predominately internalized by various endocytic routes, followed by cargo release from these vesicles inside the cells via different proposed mechanisms, and subsequent entry into the cell nucleus to promote gene expression. Compared to viral vehicles, polymeric delivery systems typically have lower delivery efficiency, and various optimization strategies can be used to improve this parameter such as, for example, changing the cationic moieties on polymers, adding targeting ligands, and installing responsive monomers, which can improve uptake efficiency and help to balance transfection efficiency and cytotoxicity. Designing novel and efficient polymer-based pDNA and RNP delivery vehicles, as well as improving the fundamental understanding of polymer-cargo complex composition and architecture on pDNA and protein loading and delivery efficiency, are necessary for advanced applications.

Polymeric vehicles are a versatile platform for the delivery of biological agents (e.g., nucleic acids) and offer numerous advantages over viral vectors by potentially enabling lower immunogenicity and production costs along with facile scalability. Cationic polymers readily complex (e.g., associate with) negatively charged biological agents through an entropically driven displacement of counterions to form interpolyelectrolyte complexes. Beyond linear cationic homopolymers, the field is being transformed through exploration of the vast chemical and architectural space afforded by recent advances in synthetic control to develop statistical and block linear copolymers, self-assembled micelles, stars, dendrimers, and cross-linked networks in an effort to overcome limitations in transfection efficiency.

In accordance with one or more examples of the disclosure, reversible addition—fragmentation chain transfer (RAFT) polymerization techniques may be used to synthesize triblock micelles, which may improve control over polymer architecture, and thus may lead to high delivery efficiency without increasing toxicity, despite increasing the density of cations within the self-assembled micelle corona. In some examples, spherical micelles may be compared to linear polymer analogues, and micelle complexes (micelleplexes) may outperform polyplexes (e.g., a plurality of linear polymer electrostatically bound to a biological agent) in delivery efficacy due to structural maintenance of the biological payload. Without wishing to be bound by any theory, the efficacy of polycationic micelles may demonstrate that polymer vehicles which induce high amine density within the corona may be advantageous for improving transfection efficiency. However, the self-assembly required to form micelles followed by a secondary formulation step can lead to challenges in scale-up. Indeed, the ability to create well-defined unimolecular architectures, comprising covalently linked cationic polymer chains clustered within a fixed volume may facilitate similar performance to cationic self-assembled micelles while granting a more complete synthetic control and reproducibility over the macromolecular architecture.

Bottlebrush (BB) polymers according to the disclosure are well-defined unimolecular architectures made up of polymer side-chains that are covalently attached to and extend radially from a central polymer backbone. In some examples, the bottlebrush polymers may be created through polymerization of macromonomers (MM). The physical properties of such systems may depend on the molar mass, degree of polymerization, and composition of both the side-chains and backbone. Orthogonal polymerization techniques may provide unique abilities to synthetically alter and isolate these and other variables.

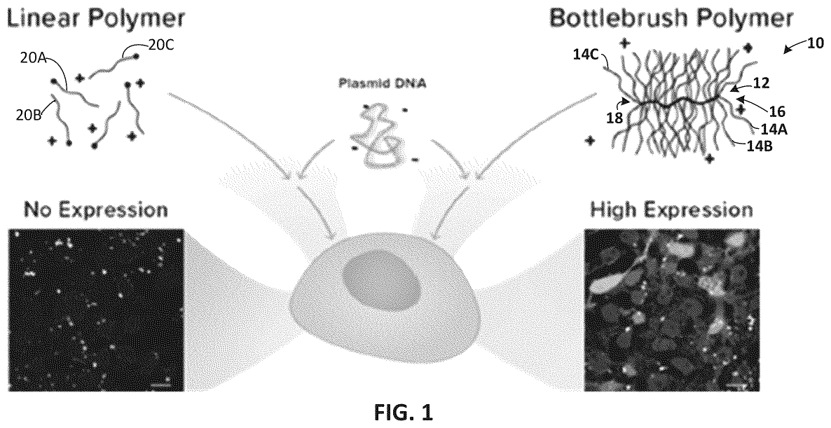

FIG. 1 illustrates the relatively higher delivery efficiency of an example bottlebrush polymer 10 over an example linear building block macromonomer 20A, 20B, (collectively “macromonomers 20”) of bottlebrush polymer 10. Bottlebrush polymer includes backbone 12 and a plurality of side-chains 14A, 14B, 14C, (collectively “side-chains 14”). For example, as illustrated in the inset picture under macromonomers associating a pDNA biological payload with macromonomers 20 may result in nearly no gene expression. However, as illustrated in the inset picture under bottlebrush polymer associating a pDNA biological payload with bottlebrush polymer 10 may result in high gene expression, indicating the efficacy of bottlebrush polymer 10 as a delivery vehicle for the biological payload.

Reversible addition-fragmentation chain transfer (RAFT) polymerization and/or ring-opening metathesis polymerization (ROMP) may be utilized in different ways to create bottlebrush polymers, such as example bottlebrush polymer 10 of FIG. 1, including by use of grafting-through techniques, grafting-from techniques, or both. Thus, orthogonal polymerization techniques may provide a synthetic platform which allows for the production of high molecular weight bottlebrush polymers while providing relative more control over dispersity, Ð, than other techniques.

Bottlebrush polymer 10 may effectively deliver many types of biological agents. In some examples, bottlebrush polymer 10 may effectively deliver small molecule therapeutics by covalent binding and/or noncovalent sequestration of the biological payload. However, bottlebrush polymer 10 may be even more desirable for delivery of larger biological payloads such as nucleic acids. Delivery of large biological payloads like plasmids (pDNA), previously not investigated, may be completed using bottlebrush systems according to the present disclosure. Unlike siRNA and oligonucleotides, pDNA payloads may present unique challenges as the long semiflexible structure may impose additional constraints on their polymeric binding partners during polymer-pDNA assembly and compaction. Moreover, unlike other nucleic acid payloads, pDNA may require delivery to the nucleus to accomplish its therapeutic function. Bottlebrush polymer 10 may have unique architectural and morphological features to overcome delivery challenges of large biological payloads including pDNA.

Bottlebrush polymer 10 may provide many advantages over other viral and non-viral delivery systems. For example, bottlebrush polymer 10 may be synthetically reproducible while offering a high molecular weight unimolecular synthetic platform to tailer isolated variables of grafting density, side-chain length, backbone length, and chemistry. Furthermore, bottlebrush polymer 10 may be densely grafted, offering physical changes to multivalency, charge, and binding. Additionally, unlike other polymeric gene delivery systems, bottlebrush polymers may offer high-aspect-ratio systems which may improve in vivo delivery outcomes and facilitate control over biodistribution profiles.

Bottlebrush polymer 10 may noncovalently bind (e.g., electrostatically bind) to biological payloads (e.g., pDNA), or be associated with the biological agent in another way (e.g., physical entanglement), forming a “bottleplex,” which may be a complex comprising bottlebrush polymer 10 and the biological agent. In this way, bottlebrush polymer 10 may be configured to wrap around a biological or other payload of interest in order to deliver the payload to a desired target, such as a particular cell. Bottlebrush polymer 10 may include backbone 12 and plurality of side-chains 14 covalently bonded to the backbone. In some examples, each side-chain 14A, 14B, and 14C of plurality of side-chains 14 may include a repeating cationic unit (e.g., a cationic monomer unit). Bottlebrush polymer 10 may be associated with a biological agent for delivery into a cell, defining a “bottleplex.” In some examples, bottlebrush polymer 10 and biological agent may be electrostatically bound. Additionally, or alternatively, the bottlebrush may be mechanically coupled to the biological agent (e.g., physically surround or partially physically surround) to form the complex. As used herein, a bottlebrush polymer that is associated with a biological agent may be electrostatically bound to the biological agent, or may be mechanically coupled to the biological agent, or both.

Bottlebrush backbone 12 may be of varying length, which in some examples may be controlled by varying the backbone degree of polymerization (N_{bb}). In some examples, N_{bb} may be from 2 to 1000, for example from about 5 to about 150, or from about 15 to about 50. In some examples, the number of side-chains 14 in the plurality of side-chains may be defined by N_{bb}, the degree of backbone polymerization. Accordingly, in some examples, the number of side-chains 14 in the plurality of side-chains may be from 2 to 1000 side-chains, for example from about 5 side-chains to about 150 side-chains, or from about 15 side-chains to about 50 side-chains. Any suitable polymer may be selected as the bottlebrush backbone. Example backbone structures may be found in ACS Nano 2020, 14, 12, 17626-17639, https://doi.org/10.1021/acsnano.0c08549, incorporated herein by reference.

As discussed above, each respective side chain 14A, 14B, 14C, may each be made up of a repeating cationic unit. Although described with respect to 2-dimethylamino ethyl methacrylate (DMAEMA) as the cationic repeating unit in the examples below, other cationic repeating units are considered. For example, other cationic monomeric repeat units may be used including one or more of amino ethyl methacrylate (AEMA), 2-(diethylamino) ethyl methacrylate (DEAEMA), and 2-(diisopropylamino)ethyl methacrylate (DIPAEMA), and mixtures and combinations thereof. Furthermore, in some examples, a cationic repeating unit may be made up of a copolymer. For example, a side chain may comprise a repeating copolymer which includes a first (meth)acryl monomeric unit with a cationic functional group R₁ and a second (meth)acryl monomeric unit with a neutral hydrophilic functional group R₂. The cationic functional group R₁ may be chosen from amino groups and alkylamino groups, and the neutral functional group R₂ may be chosen from polyethylene glycol (PEG), hydroxyl (OH), phosphorylcholine (PC), and mixtures and combinations thereof.

In some examples, each side-chain 14A, 14B, 14C may include a substantially equivalent number of repeating cationic units, such that each individual side-chain of plurality of side-chains 14 defines a substantially similar macromonomer (e.g., the same macromonomer). In some examples, side-chains 14 which include a substantially equivalent number of repeating cationic units include no more than a 15% deviation from each other in the number of repeating cationic units. In other words, each side-chain of the plurality of side-chains may have the same degree of polymerization (N_{sc}). In some examples, N_{sc} may be from 2 to 1000, for example between about 20 and about 250, or between about 30 and about 70.

Alternatively, in some examples, N_{sc} may vary among individual side-chains 14A, 14B, 14C, changing the architecture of the bottlebrush unimolecular structure. For example, still referring to FIG. 1, backbone 12 may define first end 16 and second end 18, and bottlebrush polymer 10 may define first side-chain 14A covalently bonded to first end 16 of backbone 12 and second side-chain 14C covalently bonded to second end 18 of backbone 12. In some examples, first side-chain 14A may include a greater number of repeating cationic units (i.e., a larger NO than second side-chain 14C. In this way, the bottlebrush unimolecular polymer may define a “cone” shape, which may be desirable for delivery of certain biological agents. In another example, backbone 12 may define first side-chain 14A covalently bonded to first end 16 of backbone 12, second side-chain 14C covalently bonded to second end 18 of backbone 12, and third side chain 14B covalently bonded to backbone 12 between first end 16 and second end 18. In some examples, third side-chain 14B may include a greater number of repeating cationic units (i.e., a larger NO than first side-chain 14A and second side-chain 14C. In this way, bottlebrush unimolecular polymer 10 may define a “football” shape, which may be desirable for delivery of certain biological agents. Although these first, second, and third side-chains 14 are described for illustrative purposes, more side-chains 14 may also be provided with varying lengths in order to achieve similar shapes on a larger scale, such as dozens of side-chains 14 with increasing lengths from one end of backbone 12 to the other end of backbone 12, or dozens of side-chains 14 with the shortest side-chains at the ends of the backbone with increasing lengths of side-chains towards the middle of backbone 12 such that the longest side-chains are located near the middle of backbone 12 (e.g., equidistant from first end 16 and second end 18). In other examples, other variations on the lengths of side-chains 14 along the length of backbone 12 may be used to produce different shapes of bottlebrush polymer 10. For example, more “cylindrical” shaped bottlebrushes may include a relatively longer backbone (i.e., a larger N_{bb}), which may be desirable for delivery of certain biological agents.

In some examples, suitable bottlebrush polymers for delivery of biological agents may be characterized in other ways. For example, bottlebrush polymer 10 may have a number-average molecular weight, Mₙ, from about 10 kilodaltons (kDa) to about 1000 kDa, such as from about 100 kDa to about 400 kDa. In some examples, bottlebrush polymer may define a pKₐ, which may be defined as the negative base ten logarithm of the acid dissociation constant Kₐ. In some examples, bottlebrush polymer 10 may define a pKₐ of from about 6.0 to about 9.0, such as from about 6.9 to about 7.0. In some examples, bottlebrush polymer 10 may have a zeta potential, ζ-potential, of about 10 millivolts (mV) to about 40 mV. In some examples, pKₐ and/or ζ-potential within the stated ranges may increase the efficacy of delivery of biological agents to target cells.

Although described below in the examples section primarily with respect to plasmid DNA (pDNA) as the biological agent, other biological agents are considered as the payload for delivery into a target cell. In some examples, the biological agent may be chosen from pDNA, ribonucleoprotein (RNP), and mixtures and combinations thereof. In some examples, bottlebrush polymer 10 and/or bottleplexes comprising bottlebrush polymer 10 and one or more biological agents may be included in a compound which also includes a pharmaceutically acceptable aqueous liquid carrier. Pharmaceutically accepted liquid carriers can include those liquids, emulsions, or slurries which are suitable and/or certified for inclusion in pharmaceutical compounds not as an active ingredient, but to facilitate delivery and/or transport of the active ingredient. In some examples, the compound may be delivered to a cell to repair a cell.

### Examples

A series of tests were performed to evaluate one or more aspects of some examples of the disclosure. However, the disclosure is not limited by the tests.

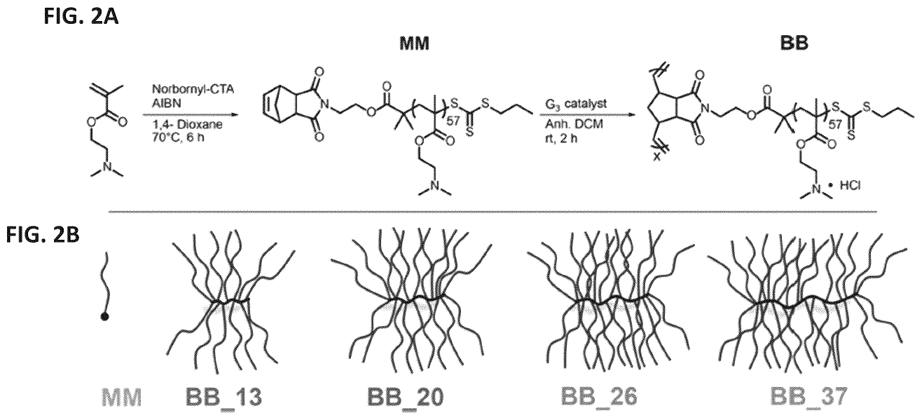

A library of cationic bottlebrush polymers were synthesized for noncovalent binding and delivery of pDNA payloads. The bottlebrush polymers synthesized had a systematic increase in backbone degree of polymerization, N_{bb}, while the side-chain degree of polymerization, N_{sc}, was maintained constant (FIGS. 2A and 2B). Cationic bottlebrush polymer-mediated pDNA delivery was evaluated by comparing unimolecular, synthetically defined bottlebrush polymers similar to bottlebrush polymer 10 of FIG. 1 to their linear building blocks, similar to macromonomers 20 of FIG. 1.

Poly(2-(dimethylamino)ethyl methacrylate) (pDMAEMA) bottlebrushes were synthesized through ring-opening metathesis polymerization to afford four bottlebrush polymers with systematic increases in backbone degree of polymerization (N_{bb}=13, 20, 26, and 37), while keeping the side-chain degree of polymerization constant (N_{sc}=57). Physical and chemical properties were characterized, and subsequently, the toxicity and delivery efficiency of pDNA into HEK293 cells were evaluated. The bottlebrush-pDNA complex (bottleplex) with the highest N_{bb}, BB_37, displayed up to a 60-fold increase in % EGFP+ cells in comparison to linear macromonomer. Additionally, a trend of increasing EGFP expression with increasing polymer molecular weight was observed. Bottleplexes (a complex including a bottlebrush polymer and an associated biological agent) and polyplexes (a complex including at least one linear macromonomer and an associated biological agent) both displayed high pDNA internalization as measured via payload enumeration per cell; however, quantitative confocal analysis revealed that bottlebrushes were able to shuttle pDNA into and around the nucleus more successfully than pDNA delivered via linear analogues. Overall, a canonical cationic monomer, such as DMAEMA, synthesized in the form of cationic bottlebrush polymers proved to be far more efficient in functional pDNA delivery and expression than linear pDMAEMA.

To synthesize the target family of bottlebrush polycations, RAFT polymerization of an amine-based monomer, 2-(dimethylamino)ethyl methacrylate (DMAEMA), with a norbornene-functionalized chain transfer agent (CTA) was conducted to yield linear macromonomers with a number-average molecular weight (M n) of 9.4 kDa.

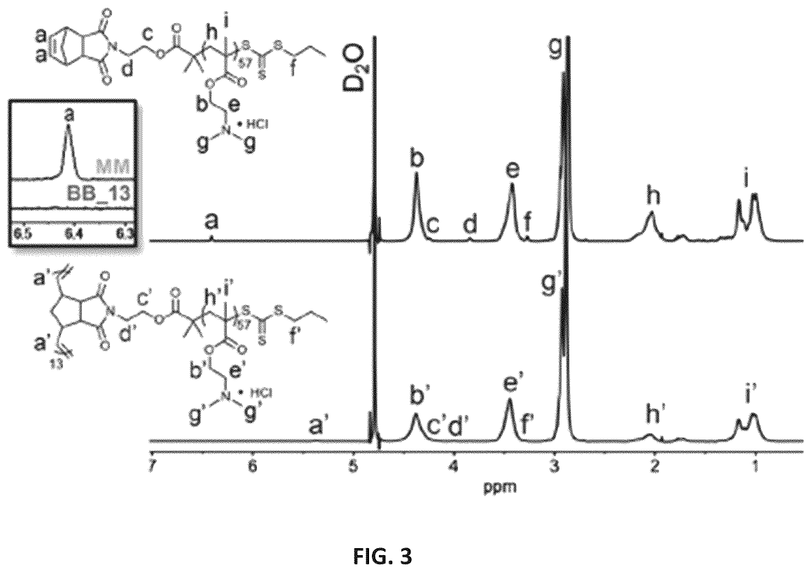

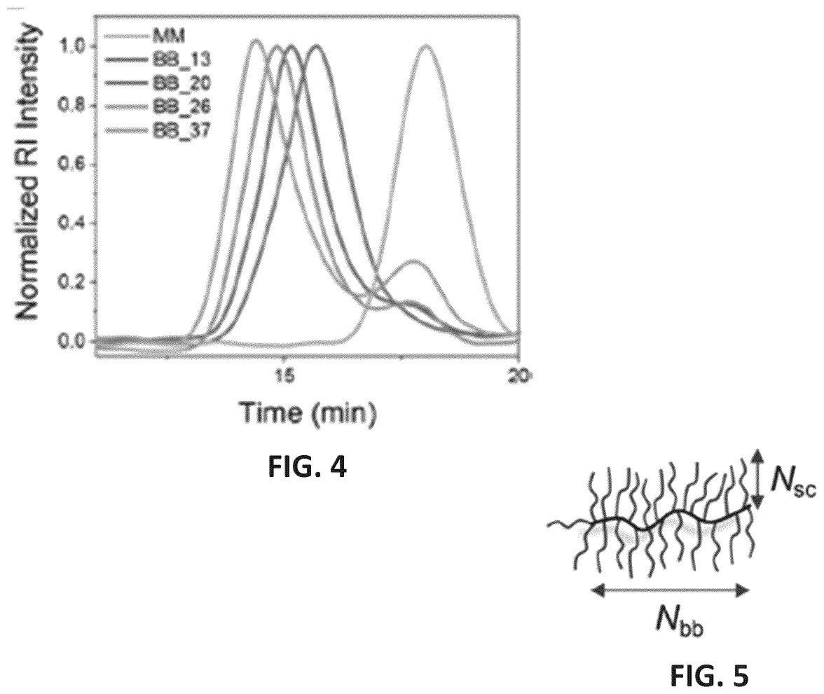

The degree of polymerization was found to be higher than theoretical, likely due to the trithiocarbonate R-group being optimized for polymerization of acrylates/acrylamides rather than methacrylates/methacrylamide. The bottlebrush polymers were synthesized through ROMP of the norbornene imide end-group on the macromonomer by varying the mole ratio of Ru-based third generation Grubbs catalyst to achieve bottlebrush polymers with systematic increases in molecular weight equating to N_{bb} repeat units of 13, 20, 26, and 37, while maintaining a relatively low dispersity (Ð≤1.33, Table 1). Size exclusion chromatography with a multiangle laser light scattering detector was used to characterize molecular weight and dispersity (FIG. 4, Table 1), and H NMR was used to identify conversion of the norbornene alkene (FIG. 3).

TABLE 1
Summary of SEC-MALS analysis of
molecular weight distributions.

| Polymer | Mₙ (kDa) | M_{w} (kDa) | Ð | N_{sc}:N_{bb} | % BB |
|---|---|---|---|---|---|
| MM | 9.4 | 11 | 1.17 | 57:1 | N/A |
| BB_13 | 124 | 143 | 1.15 | 57:13 | >95 |
| BB_20 | 184 | 220 | 1.20 | 57:20 | 90 |
| BB_26 | 246 | 309 | 1.25 | 57:26 | 90 |
| BB_37 | 344 | 458 | 1.33 | 57:37 | 81 |

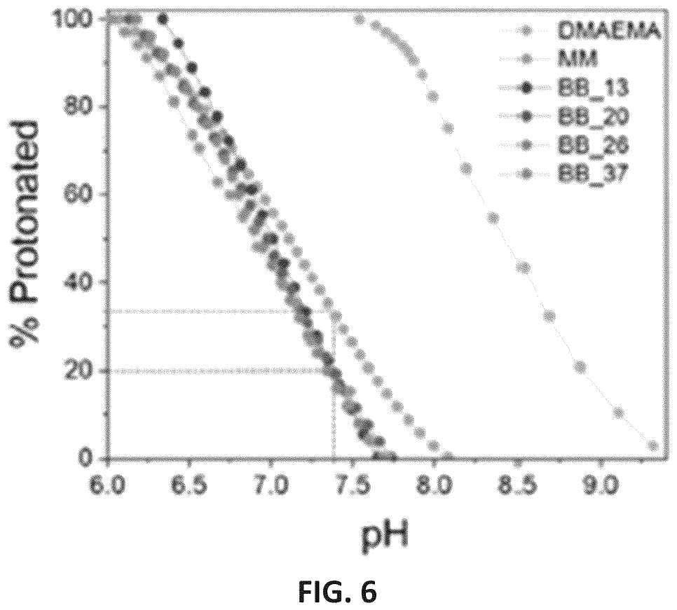

The protonation state of the multivalent bottlebrushes were characterized via acid-base titrations. The collective density of linear polymers assembled in the fixed volume of the bottlebrush architecture may lead to pKₐ changes in the physiological pH range, which may alter binding interactions between the polycationic polymers and the polyanionic pDNA payload. The pKₐ decreased by 1.4 units from monomer (DMAEMA, pKₐ=8.5) to linear polymer (macromonomer, pKₐ=7.1), which may the result of the suppression of amine ionization and proximity to charged groups upon polymerization. However, when comparing the bottlebrush polymers to the macromonomer, similar pKₐ values were found (Table 2). Uniquely, at physiological pH of 7.4, bottlebrush polymers are ˜10% less protonated than the macromonomer, indicating a dissimilarity of the charged state of the polymers (see dotted lines in FIG. 6). This property may influence charge-mediated interactions and compaction/decompaction of pDNA payloads at physiological conditions.

TABLE 2
pKa data of monomer and polymers acquired from
acid-base titration and Rh data of polyplexes
and bottleplexes formed in H2O acquired from DLS.

| Sample | pKa | Rₕ (nm) |
|---|---|---|
| DMAEMA | 8.5 | — |
| MM | 7.1 | 26 |
| BB_13 | 7.0 | 32 |
| BB_20 | 7.0 | 33 |
| BB_26 | 6.9 | 34 |
| BB_37 | 6.9 | 30 |

Polymer-pDNA/bottlebrush-pDNA complexes, termed polyplexes/bottleplexes, were analyzed for compaction ability and binding strength through gel electrophoresis, ζ-potential, and dye exclusion. Assays were completed by varying the number ratio of amines (N) in the polymer to phosphates (P) in the nucleic acid backbone (N/P ratio) to determine a minimum binding capacity. It is worth noting that with this formulation ratio the number/concentration of amines in solution are equal at each formulation ratio for each material. However, going from macromonomer to bottlebrushes of increasing molecular weight, the number of discrete polymer chains in solution decreases (as macromonomers successively covalently stitched together). Qualitative image analysis of the gel electrophoresis shift assay showed complete hindrance of pDNA migration at N/P of 1.5 and higher for all formulations. Thus, successful complexation and compaction of pDNA using bottlebrush polymers was established at low formulation ratios for all polymer architectures. When the five polymer formulations at N/P of 7.5 were measured for ζ-potential, all polymer complexes show positive charge ranging from 27 to 35 mV for the bottleplexes, while the macromonomer had an increased charge at 43 mV.

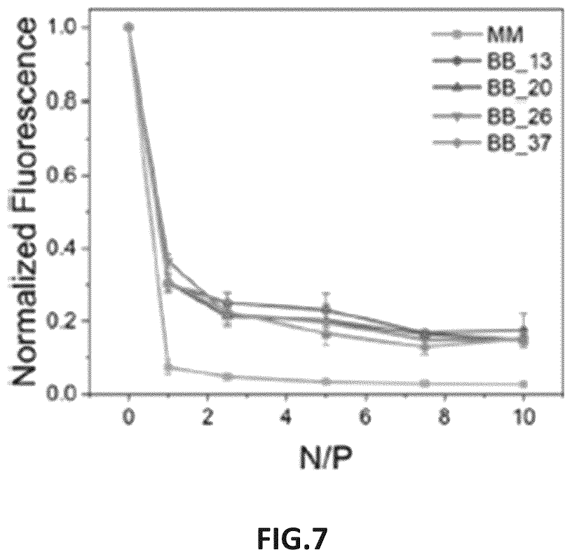

A dye exclusion assay helped gain further insight into the degree of pDNA compaction when complexed with these polycations, which involves the release of a fluorescent intercalating dye when competitive agents (the cationic macromonomer or bottlebrushes) are introduced, and no longer fluoresces when excluded from the pDNA. The macromonomer showed more displacement of the dye from pDNA in comparison to the bottlebrushes, with all four bottlebrush polymers displaying an averaged 5-fold higher fluorescence at each N/P ratio (FIG. 7). Subsequently, the gel electrophoresis shift assay showed no free pDNA above N/P 1.5, and therefore, while not wishing to be bound by any theory, it is hypothesized that the difference in fluorescence intensity between the macromonomer and the bottlebrushes may be attributed to be the degree of pDNA compaction. The bottlebrush polymers may exist as a multivalent display of linear polycations that may be more outstretched than the macromonomers, which likely exist as random coils in solution. The difference in solution structure of the bottlebrush systems may alter the binding mode, where the pDNA associates more superficially with the bottlebrush surface while the more flexible linear macromonomers may bind more intimately in the major or minor grooves, thereby displacing the intercalating dyes.

To further test the stability of these systems against competitive binding factors such as serum proteins, the polyplex/bottleplex solutions were diluted 2-fold with a 10% fetal bovine serum (FBS) solution. There was an increase in dye fluorescence after introduction of FBS, and the macromonomer has an averaged 40% increase in fluorescence, while the bottlebrushes averaged a 20% increase. Interestingly, these results showed slightly better serum stability for bottlebrushes compared to the linear analogue. Although the linear macromonomer and bottlebrush polymers have similarity in chemical functionality and pKₐ, the macromolecular structure of the bottlebrush polymer may influence the mode of pDNA binding and compaction, which may, in turn, influence intracellular release.

The size and aggregation behavior of these interpolyelectrolyte complexes may have some effect of delivery efficacy of nucleic acids into cells. Hydrodynamic radii, Rₕ, of polyplexes and bottleplexes were analyzed via dynamic light scattering after formation. The polyplexes and bottleplexes all formed similarly sized complexes in water (Rₕ˜30 nm), and did not aggregate over time, showing that macromolecular architecture may not alter the polyionic complex size during initial formulation.

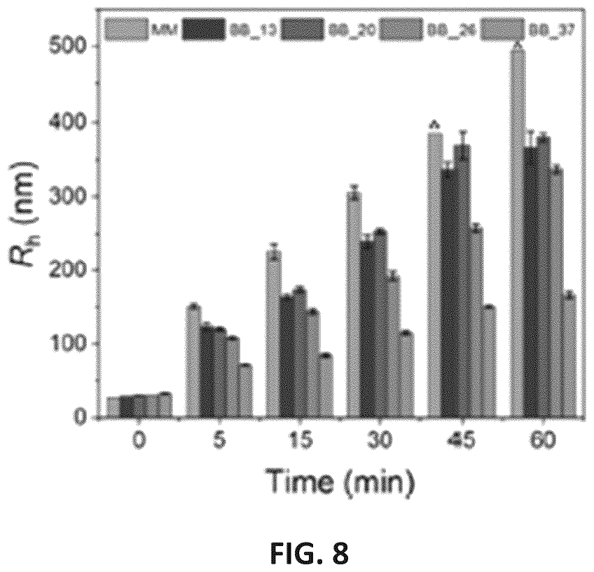

Next, to understand size and stability during transfection conditions, the formulations were further diluted in Opti-MEM and monitored for stability. Throughout the 60 min period, aggregation was observed among all five interpolyelectrolyte systems (MM, BB_13, BB_20, BB-26, BB_37, FIG. 2B). However, the macromonomer polyplexes aggregated to the largest size. A trend was observed where increasing the bottlebrush molecular weight correlated to a decrease in the aggregation behavior. After min, the bottlebrush with the longest backbone, BB_37, displayed the lowest size in Opti-MEM (Rₕ=165 nm) and had a 3-fold decrease in size compared to the macromonomer (Rₕ=495 nm, FIG. 8). The ability to tailor bottleplex size distribution and control aggregation via synthetic control with ROMP may make this delivery platform attractive for future in vivo applications.

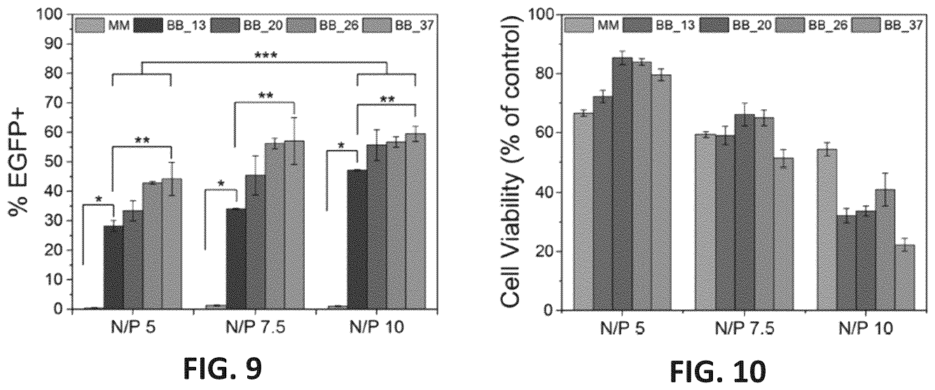

The bottlebrush polymers, along with the linear macromonomer (the building block), were directly compared to each other to determine a hierarchy of transfection ability. An enhanced green fluorescent protein (EGFP) reporter assay was used to determine the polymer vehicle's ability to deliver pDNA into cells and express the encoded protein. To test this, a solution containing the plasmid construct encoding for EGFP was mixed in formulation ratios at N/P ratios of 5, 7.5, and 10 with the macromonomer or one of the four bottlebrushes and allowed to complex for 45 minutes in water, before being further diluted in Opti-MEM and layered onto the human embryonic kidney (HEK293) cells. The differences observed in % EGFP positive cells observed by flow cytometry and fluorescence revealed a vast difference in transfection efficiency between the macromonomer and the bottlebrush formulations (FIG. 9).

Surprisingly, the macromonomer building block did not produce any notable amount of % EGFP positive cells (<1.5%), while the bottlebrush polymers ranged from 28% to 60% EGFP positive cells, depending on the N/P ratio and the backbone degree of polymerization, N_{bb}. In another study, a 25 kg/mol pDMAEMA polymer was tested on its ability to deliver pDNA to HEK293 cells, resulting in ˜5% GFP expression. The 25 kg/mol pDMAEMA is more than 2.5 times longer than the MM macromonomer, and did not provide commensurate improvement in transfection performance over the MM macromonomer. Thus, increasing the molecular weight of a linear pDMAEMA may not have significant impact on pDNA delivery performance. However, bottlebrush unimolecular architectures may improve pDNA delivery performance.

An average 1.5-fold increase of % EGFP positive cells was observed between N/P ratio of 5 and 10. At each N/P ratio, BB_37 had an average 1.5-fold increase of % EGFP positive cells compared to BB_13, suggesting that N_{bb} (and thus molecular weight) may positively correlate to transfection efficiency. Positive controls of Lipofectamine 2000 and jetPEI displayed high transfection performance, with 97% and 89% EGFP positive cells, respectively. ANOVA statistical analysis confirmed statistical significance in the performance difference between macromonomer and BB_13 as well as BB_13 from BB_37 at all three N/P ratios. Both BB_13 and BB_37 were also statistically different from themselves at polymer ratios of N/P 5 from 10, showing an increase in transfection efficiency with an increase in formulation ratio. Thus, chemical architecture may play a significant role in improving delivery efficiency, demonstrated by the up to 60-fold increase of EGFP positive cells when BB_37 was compared to the MM macromonomer.

Cell viability was measured with cell counting kit-8 (CCK-8) to understand the active metabolic process in the cell populations after transfection. Cells underwent the same transfection procedures with each formulation and were then subjected to UV-absorbance analysis of the CCK-8 dye 48 h after initial transfection. As illustrated in FIG. 10, the cell viability was found to be similarly high for all formulations at the N/P ratios of 5 and 7.5 and found to have higher cell viability than both commercial controls of Lipofectamine 2000 and jetPEI. A higher toxicity profile was found at N/P=10, with the macromonomer displaying similar levels of viable cells to jetPEI and the four bottlebrush polymers exhibiting cell viability similar to Lipofectamine 2000. This shows that the increased size and charge density of the higher ordered bottlebrush architectures were not accompanied by significantly higher levels of cellular toxicity when compared to the smaller macromonomer.

To probe whether the stark difference in transfection efficiency can be attributed to differences in cellular uptake between polyplexes/bottleplexes, internalization was measured by using fluorescently labeled cyanine 5 (Cy5)-pDNA. Cells administered with all five delivery vehicle formulations showed similarly high Cy5 fluorescence intensities (>90% positive), with little discrepancy in Cy5 intensities between the macromonomer and bottlebrush complexes (Figure S13). Thus, transfection may not be limited by cellular internalization; rather, bottleplexes may overcome some intracellular hurdle for successful expression.

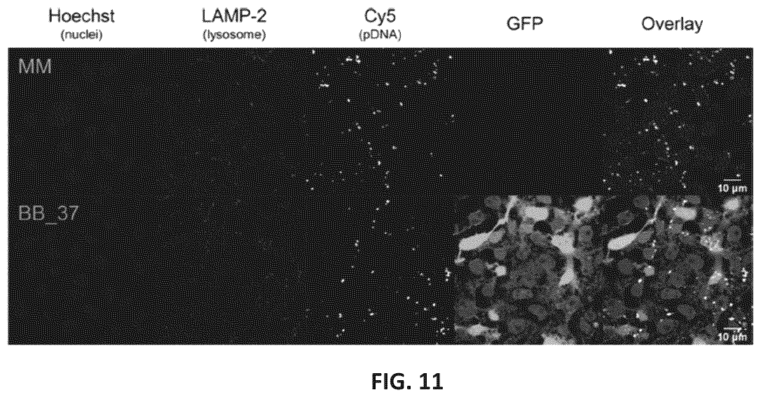

To examine the intracellular localization of polyplexes/bottleplexes, HEK293 cells were transfected with macromonomer and BB_37 complexes formulated at N/P of 7.5, and the cells were fixed 24 h following pDNA delivery. To facilitate pDNA visualization, fluorescent polyplexes were formulated by using Cy5-pDNA payloads; the outlines of lysosomal compartments were labeled by LAMP-2 and the nuclei stained with Hoechst. Consistent with observations from flow cytometry, both complexes were internalized efficiently, as indicated by the appearance of multiple Cy5 signals associated with the pDNA enumerated within cells (FIG. 11). Visually, the macromonomer (MM) and BB_37 appear similarly colocalized when stained for LAMP-2 to label lysosomes and Cy5-pDNA, and this was confirmed quantitatively with Pearson's correlation coefficients. The macromonomer displayed higher colocalization with LAMP-2 than BB_37 in EGFP positive and negative cells, and both the macromonomer and BB_37 exhibited moderate colocalization within and outside EGFP-positive cells. Despite higher cellular densities of polyplexes, payloads delivered by using the macromonomer did not culminate in EGFP expression, while in contrast very high intensities of EGFP were observed when treated with BB_37 bottleplexes, which was consistent with flow cytometry data.

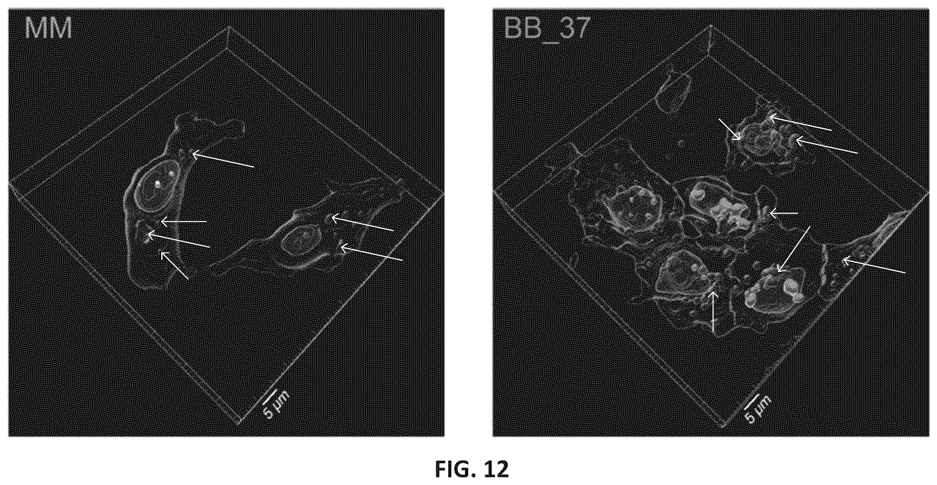

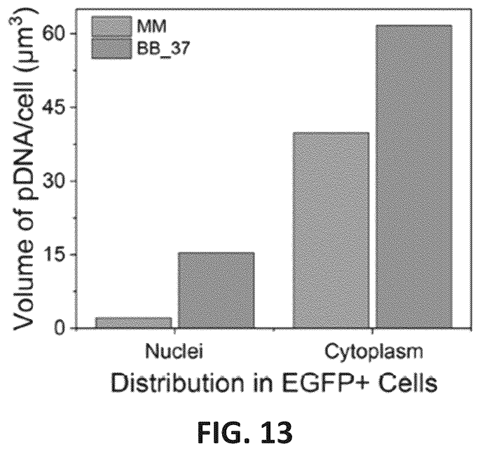

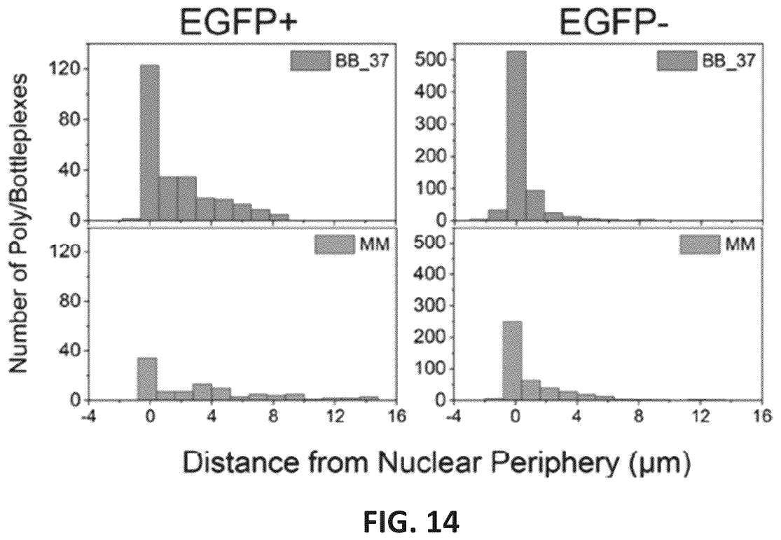

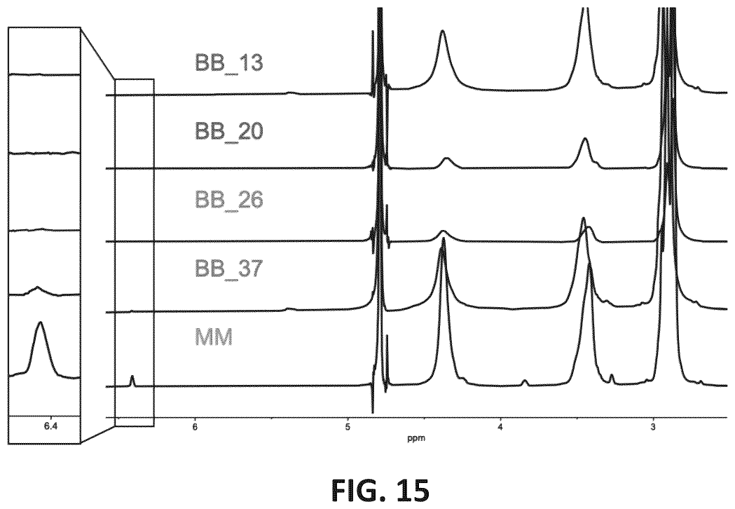

To assess the origins of gene expression disparity in functional payload delivery between the linear macromonomer polyplexes and the BB_37 bottleplexes, four to five Z-stacked confocal scans were acquired in the cell images per treatment group. From 3D reconstructions of EGFP-positive cells within each group, nuclear colocalized pDNA (white) and cytoplasmic colocalized pDNA (magenta, indicated by white arrows in FIG. 12) were able to be distinguished (FIG. 12). To quantitatively understand differences in nuclear translocation between bottleplexes and polyplexes, the volume occupied by Cy5-labeled pDNA was calculated, and distances between the Cy5-labeled pDNA to the nuclear periphery were mapped. Both sets of analyses were performed separately for EGFP-positive as well as EGFP-negative cellular populations. Among the EGFP-positive cells, more than 1.5 times more total volume of Cy5-pDNA per cell was observed in the cytoplasm and over 7-fold increase in total volume of Cy5-pDNA per cell in the nucleus when delivered by BB_37 compared to macromonomer (FIG. 6B and Table S1). When considering the distribution of the pDNA from the periphery of the nucleus among EGFP-positive and EGFP-negative cells, more pDNA was internalized and was shuttled closer to the nuclei by BB_37 (FIG. 13). Commercial controls such as Lipofectamine33 and JetPEI34 have been previously studied to show how an increased delivery of pDNA to and around the nucleus was correlated to higher gene expression. These studies further support the confocal findings that BB_37 delivered higher amounts of functional pDNA closer and to the nucleus, and may have contributed to the high transfection efficacy. Overall, quantitative confocal microscopy revealed that the distribution of pDNA payload accumulation within nuclear and cytoplasmic regions varied significantly, depending on whether they were shuttled into cells by polyplexes or bottleplexes. Bottleplexes may deliver more pDNA into cells and shuttle pDNA closer to the nucleus. The increase in total pDNA uptake via nuclear trafficking effected by BB_37 bottleplexes may contribute to the higher level of functional EGFP expression compared to the macromonomer polyplexes.

The synthesis, characterization, and application of a polycationic bottlebrush platform toward the delivery of pDNA payloads is thus disclosed. ROMP was conducted to create a series of four bottlebrush polymers with increasing N_{BB} while keeping N_{sc} fixed from one batch of linear pDMAEMA macromonomer. Although the bottlebrush polymers and the macromonomer had similar chemical functionality, pKₐ values, polyplex/bottleplex Rₕ sizes, toxicities, and internalization efficiencies, bottleplex BB_37 produced a 60-fold increase in EGFP-positive cells compared to the macromonomer building block. Flow cytometry further showed that while all bottlebrush formulations had similar internalization, increasing N_{bb} displayed on average a 1.5-fold increase in percentage EGFP-positive cells, when comparing BB_13 to BB_37. Interestingly, quantitative confocal microscopy affirmed that bottlebrush polymers delivered more pDNA into cells and also trafficked pDNA closer to the nucleus than the macromonomer, resulting in the highest transgene expression with BB_37. Although the polymers were all constructed from the same cationic repeat unit, pDNA complexes were formed in the same amine concentrations, and similar levels of cellular uptake were found across the formulations, the unimolecular architectures of the bottlebrushes overcame achieved functional pDNA delivery to the nucleus.

The following details relate to the various examples described herein. These provide some examples for the production of the bottlebrush polymers and the use thereof, but other variations for the production and use are also consistent with the compounds described herein.

### Chemical Reagents

The 2-(dimethylaminoethyl) methacrylate (DMAEMA), azobisisobutyronitrile (AIBN), generation 3 Grubbs catalyst, and DMS-sillicycle were purchased from Sigma-Aldrich (St. Louis, MO). Norbornyl CTA was prepared in a previous study by Ohnsorg et al. Ohnsorg, M. L.; Prendergast, P. C.; Robinson, L. L.; Bockman, M. R.; Bates, F. S.; Reineke, T. M. Bottlebrush Polymer Excipients Enhance Drug Solubility: Influence of End-Group Hydrophilicity and Thermoresponsiveness. ACS Macro Lett. 2021, 375-381. https://doi.org/10.1021/acsmacrolett.0c00890, incorporated herein by reference. All solvents were purchased ACS grade. Dialysis tubing (Mw cut-off=1 kDa, 40 kDa) were purchased from Spectra/Por, and were treated with 0.1 wt % ethylenediaminetetraacetic acid (EDTA) solution, and stored in a ˜0.0.05 wt % sodium azide solution. The tubing was soaked and rinsed with Milli-Q water prior to use.

### Polyplex/Transfection Reagents

The pZsGreen (4.7 and 10 kb), were purchased from Aldevron (Fargo, ND). A CCK-8 cell counting kit was purchased from Dojindo Molecular Technologies (Rockville, MD). Lipofectamine 2000, calcein violet stain, PicoGreen (Quant-iT PicoGreen, dsDNA reagent), and UltraPure ethidium bromide (10 mg/mL) were purchased from ThermoFisher Scientific (Waltham, MA). Cy-5 labelled pZsGreen plasmid was used as prepared in a previous study by Tan et al. Tan, Z.; Jiang, Y.; Zhang, W.; Karls, L.; Lodge, T. P.; Reineke, T. M. Polycation Architecture and Assembly Direct Successful Gene Delivery: Micelleplexes Outperform Polyplexes via Optimal DNA Packaging. J. Am. Chem. Soc. 2019, 141 (40), 15804-15817. https://doi.org/10.1021/jacs.9b06218, incorporated herein by reference.

### Cell Culture Reagents

Dulbecco's Modified Eagle Medium (DMEM; high glucose, pyruvate, and Glutamax supplemented), Fluorobrite DMEM (phenol red-free media), Reduced Serum Medium (Opti-MEM), Trypsin-EDTA (0.05%) with phenol red, Phosphate Buffered Saline (PBS) pH=7.4, UltraPure DNAse/RNAse-Free distilled water (DI H2O) Antibiotic-Antimycotic (100×), and Heat Inactivated Fetal Bovine Serum (HI FBS), were purchased from Life Technologies ThermoFisher Scientific (Carlsbad, CA). Cell line of human embryonic kidney cells (HEK 293T) were engineered from the laboratory of Mark Osborne at the University of Minnesota with a traffic light reporter system. To obtain a stable cell line, subcloning was performed at the Genome Engineering Shared Resource (Minneapolis, MN).

### Synthesis of MM

Homopolymer poly(2-(dimethylamino)ethyl methacrylate) (pDMAEMA, MM; Mn=9.4 kDa) was synthesized by reverse-addition fragmentation polymerization (RAFT). Commercial DMAEMA was run through basic alumina to remove trace radical scavenger (MEH). DMAEMA (15 mmol), norbornyl-CTA (1 mmol), and 2,2-Azobisisobutyronitrile (AIBN, 0.05 mmol) were dissolved in 1,4-dioxane (1.5 M, 10 mL) at a 15:1:0.05 ratio. The solution was degassed via purging with N2(g) for 45 min, and polymerized at 70° C. for 6 h. The polymerization reaction was slowed by cooling the solution in an ice bath, then quenched with exposure to air. The obtained polymer was purified in a 1 kDa dialysis bag in MeOH to nanopure H2O and freeze-dried. The final polymer was a yellow solid resulting in MM. Characterization was completed by 1H NMR spectroscopy (Mn, NMR=9.2 kDa, N_{sc}, NMR=48), and by DMF (0.05 M LiBr) SEC-MALS, as shown in FIG. 2. (Mₙ=9.4 kDa, Mw=11 kDa, Ð=1.17, N_{sc},SEC=57).

### Ring Opening Polymerization General Synthesis of Bottlebrush Polymers

All bottlebrush polymers of pDMAEMA were synthesized via a general ring opening metathesis polymerization (ROMP) procedure, however each reaction varied in amount of Grubbs generation 3 (G3) catalyst. In a nitrogen filled glovebox, MM was dissolved in DCM and allowed to stir for 10 min. G3 catalyst was prepared in a 10 mg/mL solution and then added to the vial containing MM to give a final concentration of around 10 mM in DCM. The reaction mixture was stirred at room temperature for 2 hours before quenching outside the glovebox with excess ethyl vinyl ether (0.1 mL), diluted with 5 mL DCM and stirred for an additional 15 min. SiliaMetS DMT metal scavenger was then added and stirred at room temperature overnight. The reaction mixture was filtered, concentrated in vacuo, dissolved in 1:3 (4 mL) MeOH:1 M HCl, dialyzed against nanopure H2O via a 40 kDa bag, and freeze dried to yield a light-white powder. Characterization was completed by 1H NMR spectroscopy, and by DMF (0.05 M LiBr) SEC-MALS, as shown in FIG. 3.

### ROMP Condition Specifics for BB_13, BB_20, BB_26, and BB_37

BB_13: Macromonomer (100 mg, 0.0111 mmol) underwent ROMP with G3 (1 mg, 11.4 104 mmol) in DCM (0.805 mL, 14 mM). (Mₙ=124 kDa, M_{w}=143 kDa, D=1.15, N_{bb}=13).

BB_20: Macromonomer (124 mg, 0.01378 mmol) underwent ROMP with G3 (0.64 mg, 7.3 104 mmol) in DCM (1.378 mL, 10 mM). (Mₙ=184 kDa, M_{w}=220 kDa, D=1.20, N_{bb}=20).

BB_26: Macromonomer (105 mg, 0.01164 mmol) underwent ROMP with G3 (0.34 mg, 3.8 104 mmol) in DCM (1.164 mL, 10 mM). (Mₙ=245 kDa, M_{w}=309 kDa, D=1.25, N_{bb}=26).

BB_37: Macromonomer (111 mg, 0.01237 mmol) underwent ROMP with G3 (0.19 mg, 2.2 104 mmol) in DCM (1.237 mL, 10 mM). (Mₙ=344 kDa, M_{w}=458 kDa, D=1.33, N_{bb}=37).

Offline Batch Mode Measurement of ∂n/∂c of Macromonomer and Bottlebrush

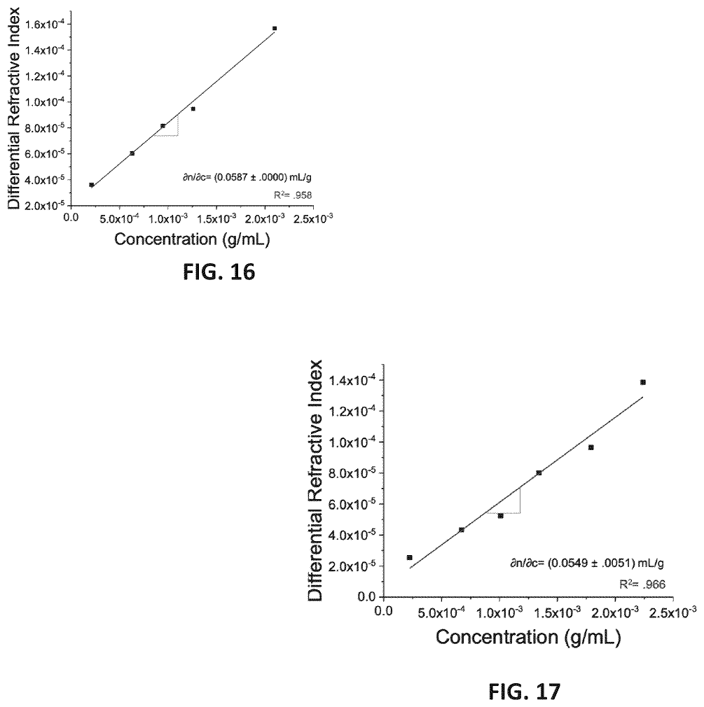

A stock solution of polymer was made at in DMF (LiBr 0.05 M). Five dilutions were made with DMF (LiBr 0.05 M) to achieve an order of magnitude difference with a concentration range of 0.21-2.1 mg/mL for MM and 0.24-2.4 mg/mL for BB_13. Samples were injected at a flow rate of 0.1 mL/min into a Wyatt Optilab T-rEX refractive index detector (25° C., λ0=660 nm). Refractive indices were measured at each concentration and the ∂n/∂c is determined from the slope. MM: ∂n/∂c=0.0587 BB_13: ∂n/∂c=0.0549. Illustrations of the Differential Refractive Index for MM and BB_13 are illustrated in FIGS. 16 and 17, respectively.

### pKₐ Titration

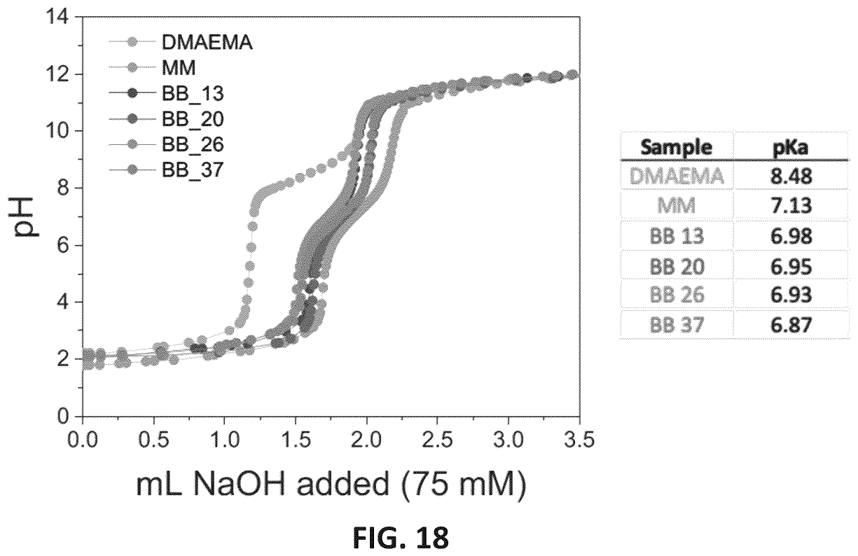

FIG. 18 illustrates titration data of the DMAEMA macromonomer MM and four bottlebrush polymers. Monomer and polymers MM, BB_13, BB_20, BB_26, and BB_37 were dissolved at 1 mg/mL in 30 mM HCl using an auto-titrator, Orion star T901 pH titrator (Thermo Fisher, Waltham, MA) The prepped solutions were titrated with 75 mM NaOH.

### Biological Binding Characterization and Delivery

### Cell Culture Procedures

The HEK 293T cell line was cultured in high glucose DMEM with added 10% HI-FBS and 1% Antibiotic/Antimicrobic. The incubator was set to 37° C. with 5% CO2 and under humidified atmosphere. Cell confluency was monitored, and cells were passaged as needed. Cells were plated in a 24-well plate format at a density of 50,000 cells/mL.

### In Vitro Cell Transfection Using Polyplexes Procedures

Quantification of transfection efficiency of HEK293 cells delivering pDNA encoding for EGFP were measured via flow cytometry. HEK293 cells were plated in a 24-well plate at a density of 50,000 cells/mL. After 24 hours, polyplexes were prepared in H2O by adding 170 μL polymer to 170 μL pDNA (0.0211 g/mL) at various molar ratios of polymer to get N/P ratios of 5:1, 7.5:1, and 10:1. Polyplexes were allowed to form at room temperature for 40 min. Twice the volume of (680 μL) of Opti-MEM was added to the polyplexes immediately before addition to cells. Media was aspirated from the well plate before addition of polyplex sample. Each polyplex was split into triplicate adding 300 μL to each well. Well plate remained on the bench top for 40 min before placing into the 37° C. incubator. 4 h after initial transfection, 1 mL of DMEM (10% HI-FBS) was added to each well. Media was further aspirated 24 hours after initial transfection, and fresh DMEM (10% FBS, 1 mL) was added to each well. The cells were analyzed for CCK-8 and flow cytometry analysis 48 hours following initial transfection.

### Dynamic Light Scattering (DLS) Data

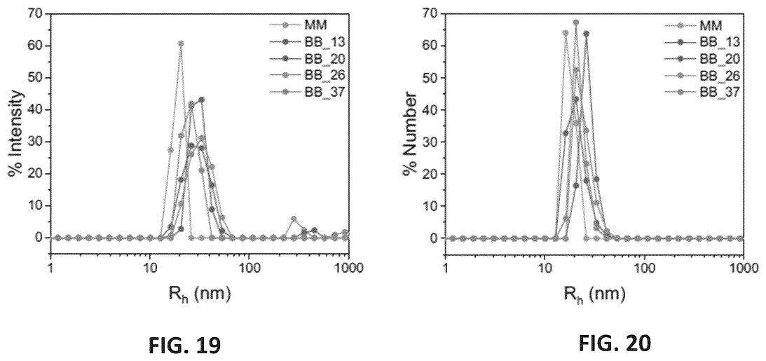

FIGS. 19 and 20 illustrate dynamic light scattering data (DLS) of polyplex and bottleplexes of MM, BB_13, BB_20, BB_26, and BB_37. The polyplex and bottleplexes were formed at N/P ratio of 7.5 and were measured in order to better understand the hydrodynamic radius (Rₕ). All water was pre-filtered through a 0.2 μm GHP syringe filter and prepared to run in a high throughput DLS DynaPro Plate Reader III (Greiner Bio One GmbH, SensoPlate, 655892, Wyatt Technology, Santa Barbara, CA). For high-throughput DLS, samples were transferred into a glass bottomed 96-well DLS plate. The well plate was placed in the DynaPro Plate Reader III and equilibrated at 25° C. Polyplexes were prepared in H2O by adding 100 μL polymer to 100 μL pDNA (0.02 μg/mL) at a molar ratio of polymer to achieve N/P ratios 7.5:1. Wells containing samples of interest were analyzed using automated measurements after letting the polyplex form for 40 min in the well. For each measurement, ten acquisitions were recorded with an acquisition time of 5 seconds each. For aggregation studies, a 2-fold volume increase of Opti-MEM was then added to these wells and DLS measurements were taken over an hour timespan.

### Gel Electrophoresis

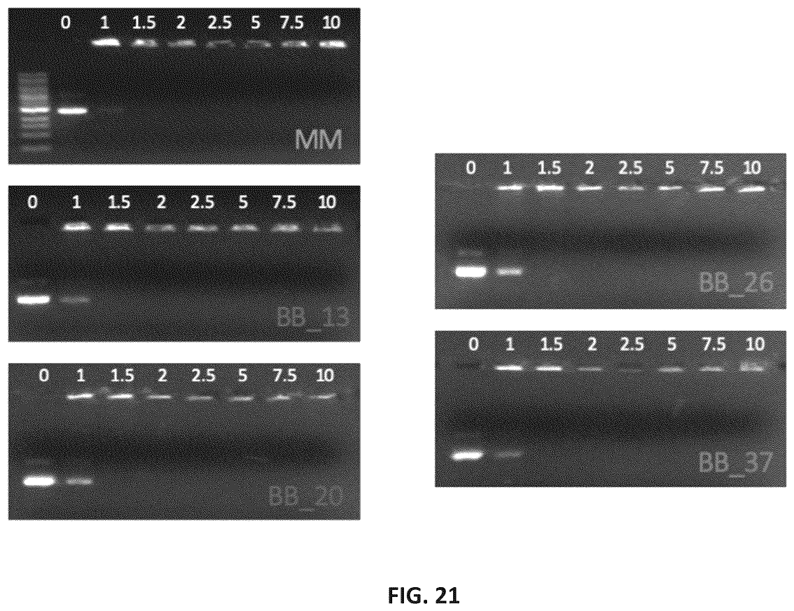

FIG. 21 illustrates gel electrophoresis of the polyplex and four bottleplexes showing complete binding at an N/P ratio of 1.5 and greater. Polyplexes were prepared in H2O by adding 10 μL polymer to 10 μL pDNA (0.0211 g/mL) at various molar ratios of polymer to get N/P ratios of 0:1, 1:1, 1.5:1, 2:1, 2.5:1, 5:1, 7.5:1, and 10:1. Polyplexes were allowed to form at room temperature for 40 min. Loading buffer was prepared by mixing 20 μL H2O with 4 μL loading buffer (6×). Of that solution, 20 μL of the 1× loading buffer was added to the 20 μL polyplex solution, mixed and then added to the wells in the gel (20 μL). The 0.6% agrose gel contained 1 μg/mL ethidium bromide/TAE buffer. The electrophoresis was run for 60 min at 70 mV. The gel was visualized using a standard UV transilluminator box (Fotodyne, IL, USA).

### Dye Exclusion Assay

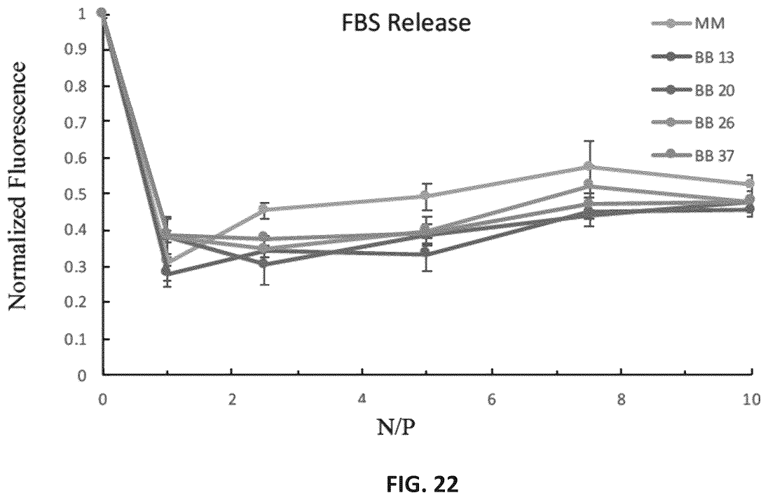

FIG. 22 illustrates dye exclusion data showing the amount of fluorescence of PicoGreen before and after the addition of 10% FBS solution. To understand binding strength of polymers to pDNA, dye exclusion assay with PicoGreen was run. PicoGreen dye was incubated for 15 min with pDNA (0.02 μg/mL) was diluted to a final concentration with a ratio of 1:200 in H2O. Polyplexes were prepared in H2O by adding 170 μL polymer to 170 μL Pico Green-pDNA (0.02 μg/mL) at various molar ratios of polymer to get N/P ratios of 0:1, 1:1, 2.5:1, 5:1, 7.5:1, and 10:1. Polyplexes were allowed to form at room temperature for 40 min and then split into triplicate by adding 100 μL to a 96 well plate. To test serum stability, a 10% FBS containing solution of Fluorobrite was added in a 2-fold volume increase (200 μL) and further measured. Fluorescence was measured (excitation: 485 nm, emission: 528 nm) using a Synergy H1 Hybrid Reader (BioTek, Winooski, VT).

### Zeta Potential

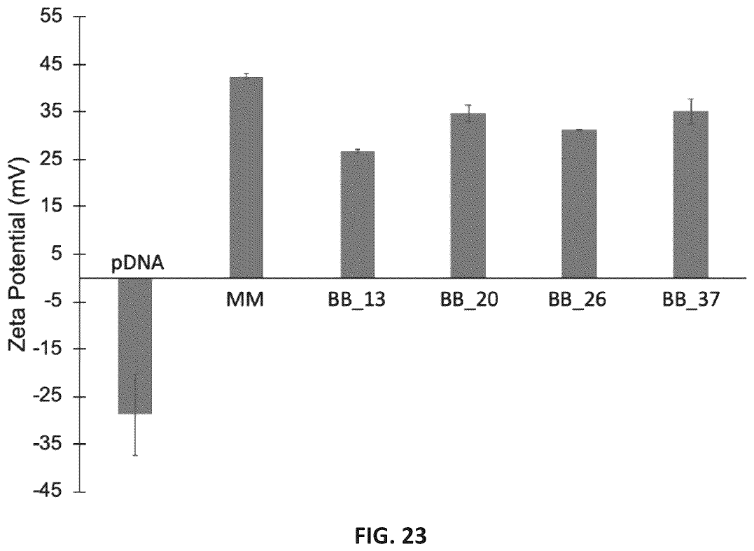

FIG. 23 illustrates the zeta potential (ζ-potential) of pDNA, MM polyplex, and four example bottleplexes at an N/P ratio of 7.5. The polymer and pDNA samples were prepared at 0.01 mg/mL of pDNA, as discussed above. All polymer samples were measured at an N/P ratio of 7.5. Formulations were added to a folded capillary cell with gold plated cells (DTS1070) and the zeta potential was measured by a Malvern Zetasizer Nano ZS. The zeta potential was measured via electrophoretic mobility with the Smoluchowski equation.

### EGFP Reporter Assay Measurement Using Flow Cytometry

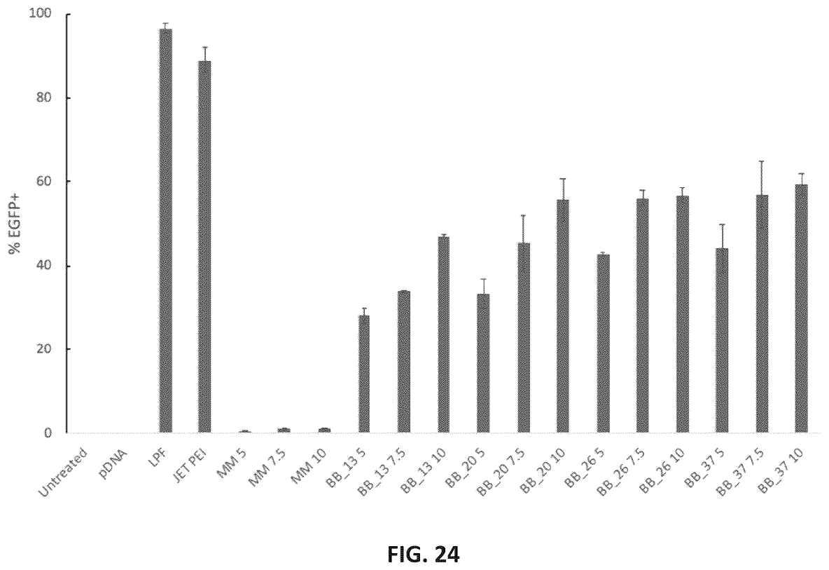

FIG. 24 illustrates the percent EGFP-positive (EGFP+) cells transfected with MM, BB_13, BB_20, BB_26, and BB_37 at N/P ratios of 7.5, 10, and 12.5. Following 48 hours after initial transfection, cells were harvested for flow cytometry to quantify percentage of EGFP positive cells. To harvest cells, media was aspirated, HEK293 cells were trypsinized (250 μL), and added to a V-shaped 96-deepwell plate to be centrifuged at 4° C. The supernatant was aspirated and cell pellet was resuspended with calcein violet live-cell stain in PBS with 10% FBS. Cells incubated with the cell stain for 30 min on ice without light before measuring via flow cytometry for EGFP. 405 nm (calcein violet) and 488 nm (GFP) lasers were used on the flow cytometer (ZE5, Biorad, Inc., CA, USA). At least 5,000 events were collected for every treatment in triplicate.

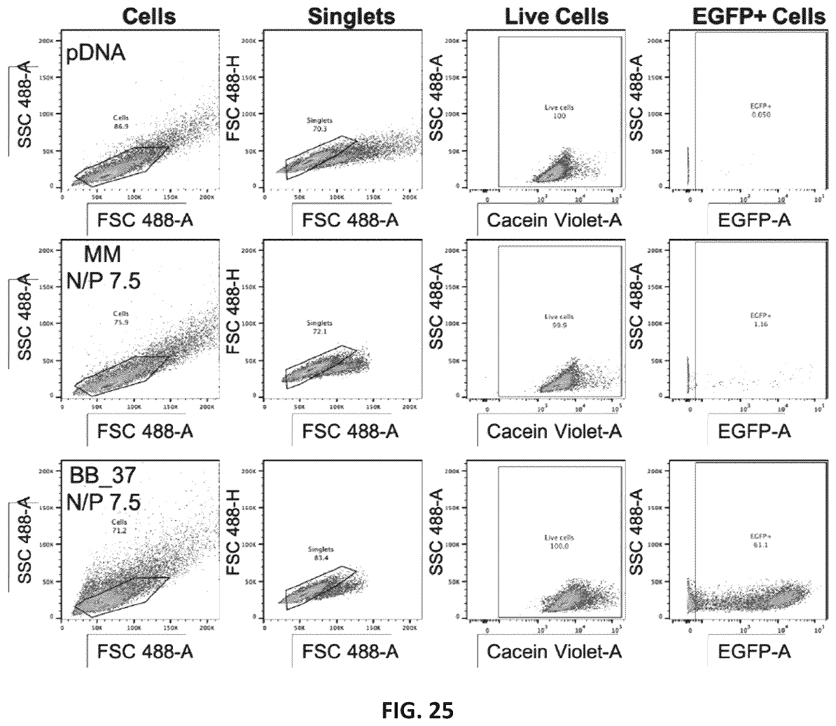

FIG. 25 illustrates the gating scheme used for identifying the amount of EGFP+ cells. Three representative examples are shown in pDNA, MM at N/P 7.5, and BB_37 at N/P 7.5.

### Cell Viability Assay Using CCK-8

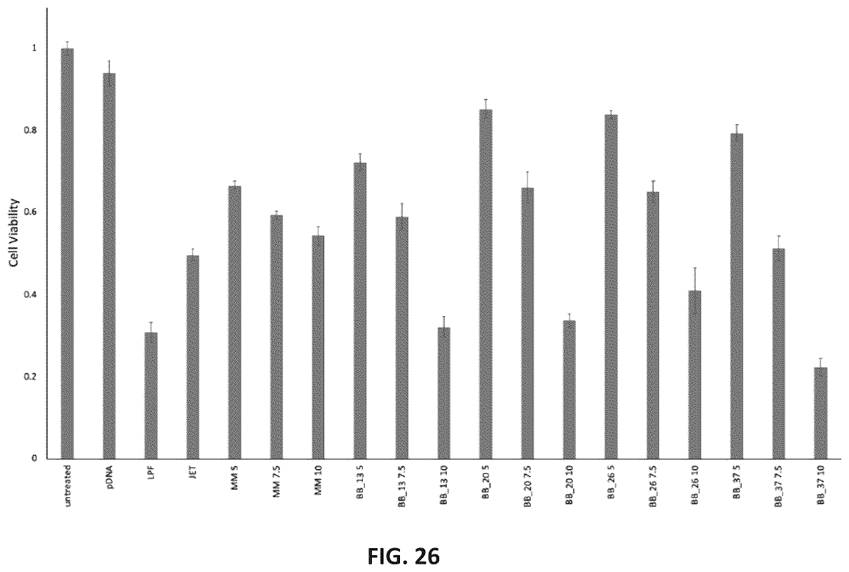

FIG. 26 illustrates the viability of cells transfected with MM, BB_13, BB_20, BB_26, and BB_37 at N/P ratios of 5, 7.5, and 10. Cell counting kit-8 (CCK-8) was used to measure cell viability after transfection. Transfection procedures were carried out as written in in vitro cell transfection using polyplex section. 48 hours post initial transfection, media was aspirated. Fluorobrite (1 mL) containing 10% FBS was mixed with CCK-8 dye (40 μL) and added to each well to incubate for 2 h. After incubation, 200 μL of the supernatant was removed and placed in a 96-well plate and absorbance was measure at 450 nm using a Synergy H1 Hybrid Reader (BioTek, Winooski, VT). Untreated cells were normalized to 100% cell survival.

### Cy5 Internalization Study

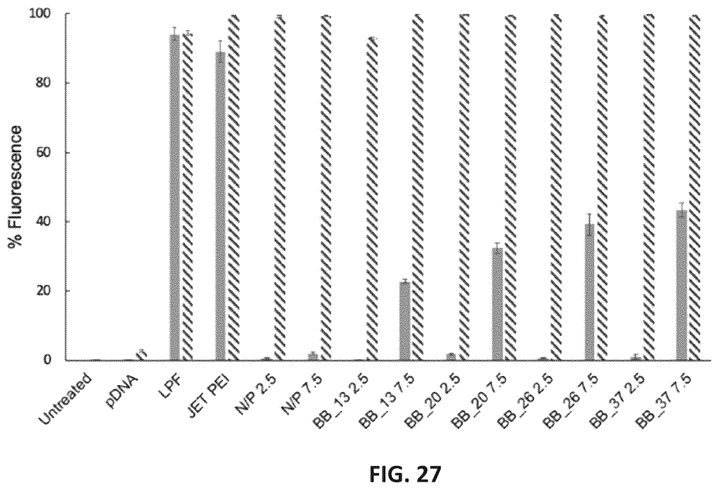

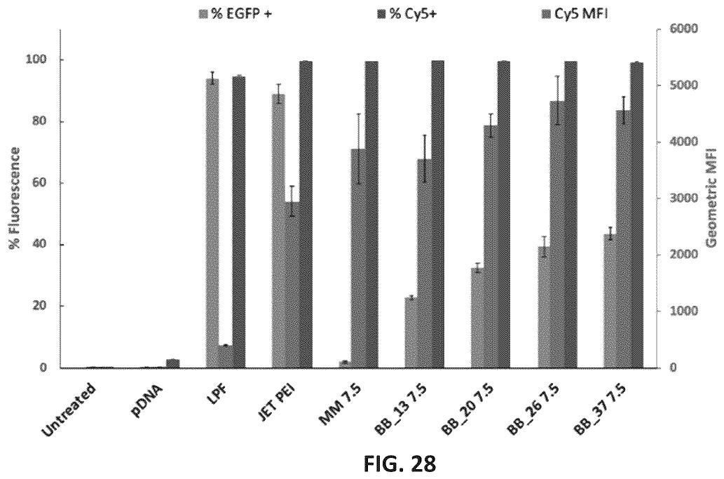

FIG. 27 illustrates the percent Cy5 and EGFP+ cells at N/P ratios of 2.5 and 7.5, with EGFP illustrated in solid and Cy5 illustrated as dashed. FIG. 28 illustrates the percent Cy5 and EGFP cells and geometric MFI of Cy5 transfected with MM, BB_13, BB_20, BB_26, and BB_37 at N/P ratios of 7.5 at 24 h timepoint. In order to determine internalization of pDNA, cells were prepped on 24-well plates as stated above. Cells were worked up for flow cytometry 24 hours after initial transfection. Media was aspirated, trypsinized (250 μL) and added to a V-shaped 96-deepwell plate to be centrifuged at 4° C. The supernatant was aspirated and then the resuspended and incubated with cell scrub for 10 min. Samples were topped with PBS, pelleted, aspirated and washed once more with PBS. The supernatant was aspirated and then the resuspended with calcein violet live cell stain in PBS with 10% FBS. Cells incubated with the cell stain for 30 min on ice without light before measuring via flow cytometry. Cells were analyzed for both Cy5 and EGFP. 405 nm (calcein violet), 488 nm (GFP) and 633 nm (Cy5) lasers were used on the flow cytometer (ZE5, Biorad, Inc., CA, USA).

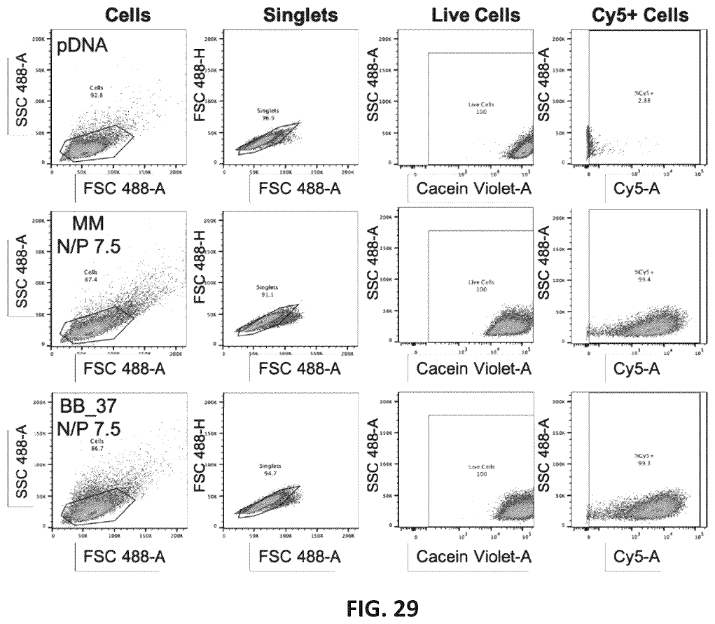

FIG. 29 illustrates the gating scheme used for identifying the amount of Cy5-positive (Cy5+) cells. Three representative examples are shown in pDNA, MM at N/P of 7.5, and BB_37 at N/P of 7.5.

### 2D Microscopy Images

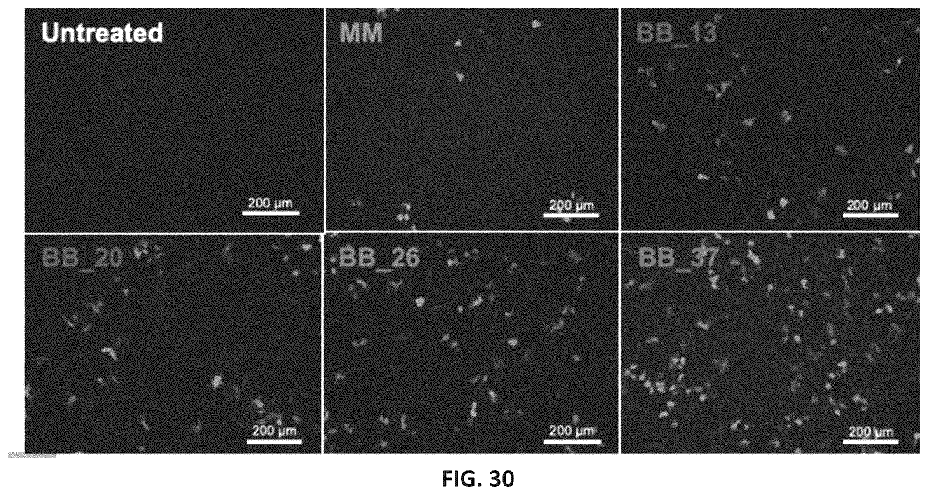

FIG. 30 illustrates microscope images of HEK293 cells 24 h after initial transfection with a GFP filter to image EGFP positive cells. Images show the change in EGFP positive cells when comparing cells that were not transfected (untreated) as well as transfected with polyplex and the four bottleplexes at an N/P of 7.5. 2D images were taken via \ an EVOS Digital Microscope (AMG Life Technologies; Grand Island, NY). Images were taken at a 24 h timepoint in representative wells for each polymer sample. EGFP cells (ZsGreen (λₑₓ=470/40 nm, λₑₘ=525/50 nm)) were pictured using an using a GFP filter cube.

### 3D Confocal Microscopy

### Cell Prep for Confocal Microscopy

Transfection procedures were performed using the typical procedures listed above with Cy5 labeled pDNA. Post transfection, 24 h, HEK293 cells were thoroughly washed using cold PBS and immersion-fixed within a cold fixing solution (PBS, 4% formaldehyde) for 15 min. Thereafter, cells were washed twice with blocking buffer (PBS, 0.2% gelatin, 5% BSA, and 0.1% Triton-X). Cells were immersed in a solution of anti-LAMP2 primary antibody (Abcam ab25631, Cambridge, MA) diluted to 1:100 in the blocking buffer, for an hour at ambient temperature for proper cellular permeabilization. Cells were washed four times in 5 min increments each (PBS, 0.1% Triton-X). Following washing, a 30 min incubation period with a secondary staining antibody (Invitrogen catalog #A11003, Waltham, MA) diluted to 1:1000 in blocking buffer was added. Further, cells were counterstained with Hoechst 3342 and then washed three times in 5 min increments (PBS, 0.1% Triton-X). Coverslips were lastly rinsed with ultra-pure water, followed by being dried and mounted on Prolong Glass (Thermo Fisher, Waltham, MA). Cells cured at room temperature with the absence of light for two days. Samples were sealed with nail polish, and further imaged following the microscope set up detailed by Kumar et al. Kumar, R.; Le, N.; Tan, Z.; Brown, M. E.; Jiang, S.; Reineke, T. M. Efficient Polymer-Mediated Delivery of Gene-Editing Ribonucleoprotein Payloads through Combinatorial Design, Parallelized Experimentation, and Machine Learning. ACS Nano 2020, 14 (12), 17626-17639. https://doi.org/10.1021/acsnano.0c08549, incorporated herein by reference.

### Laser Scanning Confocal Analysis

Every sample was imaged on an Olympus laser-scanning confocal microscopy system equipped with an upright BX2 microscope with a PLAPON 60× oil objective (NA 1.42) plus a 488 nm Argon laser, 543 nm HeNe laser, and 405 nm plus 635 nm solid-state diode lasers. The system was controlled with Olympus FluoView FV1000 software, version 4.1.15. Laser power was set to 43% for the 405 nm laser, 10% for the 488 nm laser, 70% for the 543 nm laser, and 10% for the 635 nm laser. Laser emission was passed through a 405/488/543/635 dichroic mirror and fluorescence emission was collected with emission filters in the ranges of 430-470 nm for Hoechst signal, 505-525 nm for EGFP signal, 560-660 nm for AlexaFluor568 signal, and 655-755 nm for Cy5 signal. Voltage settings for the photomultiplier tube detectors were 408 V for the blue detector, 336V for the green detector, 581 V for the orange-red detector, and 348 V for the far-red detector. 800 by 800-pixel images were collected with a pixel dwell time of 8 pec. Images were collected at the Nyquist sampling rate for the 60× oil objective, giving voxel dimensions of 91 nm laterally and 440 nm axially.

### Spectral Unmixing

The DNA particles were labeled with Cy5 and while the cell nuclei were labeled with Hoechst. Due to the use of Hoechst, DNA within the polyplexes was inadvertently labeled, requiring spectral unmixing of Cy5 and Hoechst signals. Spectral unmixing was performed with the ROI method in Nikon Elements Analysis software (version 5.21.01).

### Quantitative Image Analysis

DNA particle counts and particle volume measurements in the nucleus and cytoplasm compartments were measured in Imaris software, version 9.7.1 (Bitplane, Zurich, Switzerland). The Cells module was employed to segment Hoechst-labeled nuclei and EGFP-labeled cells. The EGFP+ nuclei were sufficiently bright that the EGFP channel could be used to segment nuclei rather than the Hoechst-labeled nuclei. Nuclei were segmented by seed points with a diameter of 6 μm and thresholded at 268 intensity. EGFP+ cells were thresholded at 98 intensity and segmented by allowing only one nucleus per cell. Both EGFP+ nucleus and cell renderings were exported as separate surfaces in the Surface module. Next the EGFP+ nuclei and EGFP+ cells were masked in order to facilitate segregation of the Cy5+ DNA particles into separate surfaces to distinguish between cytoplasmic and nuclear DNA particles. Once in separate surfaces, the nuclear and cytoplasmic DNA particles could be counted, and particle volume measured. The particle volume inside nuclei and cytoplasm were summed and the proportion of DNA particles inside the EGFP+ nucleus versus the cytoplasm were calculated.

The EGFP-negative (non-EGFP) nuclei were treated as a population rather than segmented. The EGFP+ cells were masked and removed from the image so that only non-EGFP nuclei and DNA particles remained. Again, the particles inside non-EGFP nuclei were counted and their volumes measured. The counts and volume of particles outside non-EGFP nuclei were measured as well. Again, the proportions of DNA inside non-EGFP nuclei versus extranuclear particles were calculated.

TABLE 3
Total volume (μm3) of pDNA particles within EGFP(+/−) cells
when delivered by MM and BB_37

| | pDNA | pDNA | pDNA | pDNA |
|---|---|---|---|---|
| | Volume | volume | Volume | volume |
| | in EGFP+ | in EGFP+ | in EGFP− | in EGFP− |
| Polymer | Nuclei | Cytoplasm | Nuclei | Cytoplasm |
| MM | 27.9 | 517.5 | 1186.5 | 886.8 |
| BB_37 | 353.6 | 1418.5 | 3410.9 | 859.6 |

### Lysosome and Cy5-pDNA Colocalization

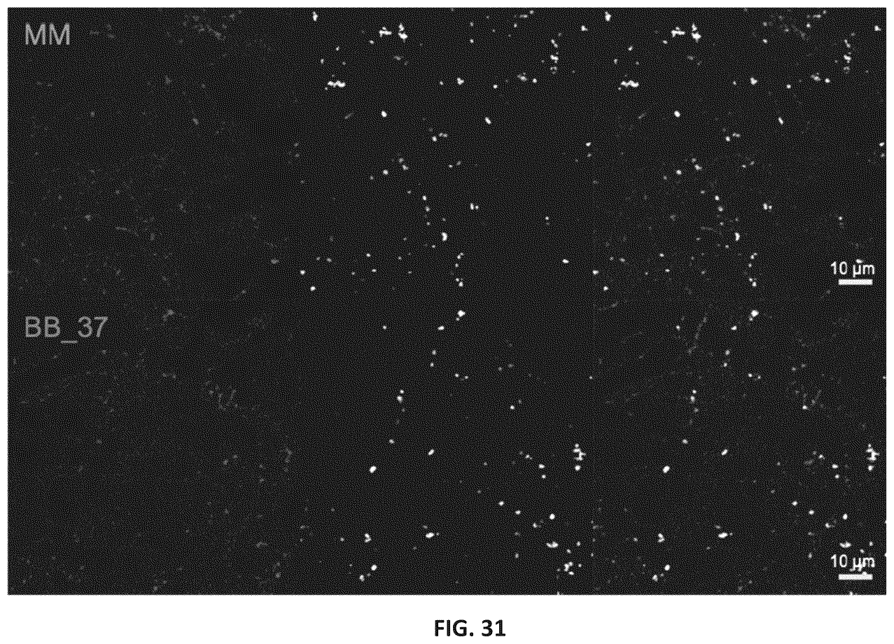

FIG. 31 illustrates confocal 2D images comparing MM and BB_37 by tracking Cy5 labeled pDNA internalized into HEK293 cells, cells fixed at 24 h. Confocal microscopy observations: lysosomal LAMP-2 stain, Cy5-pDNA fluorescent tag. Same imaging methods as previously written. All images were processed and colocalization between lysosomes and pDNA particles (white, left panel) quantified with Imaris software, version 9.7.2 (Bitplane, Zurich, Switzerland). First background was subtracted in all channels followed by masking all voxels outside EGFP-expressing cells and inside the nuclei of EGFP-expressing cells such that all voxels within these regions were set to zero intensity. The remaining voxels with signal were present in the EGFP cytoplasm. These voxels were thresholded with Imaris software and three-dimensional Pearson's correlation coefficients were quantified. The colocalizing voxels within EGFP cytoplasm were colored (arrows, right panel). To estimate lysosome colocalization outside EGFP-expressing cells, the EGFP-expressing cells were masked and set to zero intensity. All voxels of non-EGFP-expressing cells were thresholded and three-dimensional Pearson's correlation coefficients calculated. Colocalizing voxels in cells without EGFP were colored (arrows, right panel).

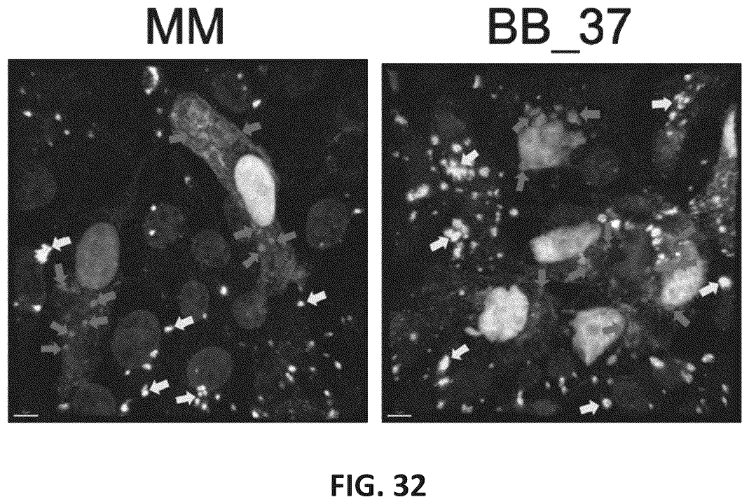

FIG. 32 illustrates colocalization of Lamp-2-stained lysosome and Cy5 tagged pDNA. Lysosomes (dark), pDNA particles (light), colocalized voxels of lysosome and pDNA in EGFP-positive cells (grey arrows), and colocalized voxels lysosome and pDNA in EGFP-negative cells (light arrows). Examples of colocalized voxels highlighted with arrows.

TABLE 4
Pearson's coloration coefficient for colocalized volume
of the overlap of the lysosome and the pDNA voxels and
Tukey HSD p-values evaluated by ANOVA analysis.

| | Pearson's | Tukey | Pearson's | Tukey |
|---|---|---|---|---|
| | Correlation | HSD | Correlation | HSD |
| | Coefficient in | p-value | Coefficient in | p-value |
| Polymer | EGFP+ | (EGFP+) | EGFP− | (EGFP−) |
| MM | .440 | .0321 | .516 | .0010 |
| BB_37 | .216 | | .225 | |

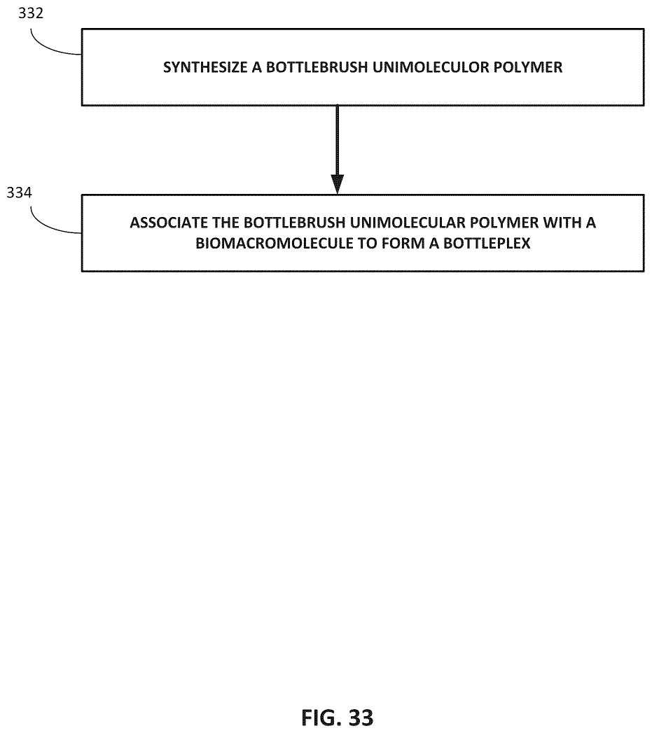

FIG. 33 is a flowchart illustrating an example technique in accordance with one or more examples of the present disclosure. The technique of FIG. 33 is described with reference to FIG. 1. The technique of FIG. 33 includes synthesizing a bottlebrush unimolecular polymer 10 (332). Bottlebrush polymer 10 includes backbone 12 and side-chains 14. Each respective side-chain 14A, 14B, 14C of side-chains 14 comprises a repeating cationic unit (e.g. a monomer).

The technique of FIG. 33 also includes associating bottlebrush polymer 10 with a biomacromolecule to form a bottleplex (334). In some examples, the biomacromolecule may include pDNA, RNP, or combinations thereof. In some examples, associating bottlebrush polymer 10 with the biomacromolecule may include one or more of electrostatically binding, mechanically coupling, or combinations thereof. The resulting bottleplex may be a complex comprising bottlebrush polymer 10 and the biomacromolecule.

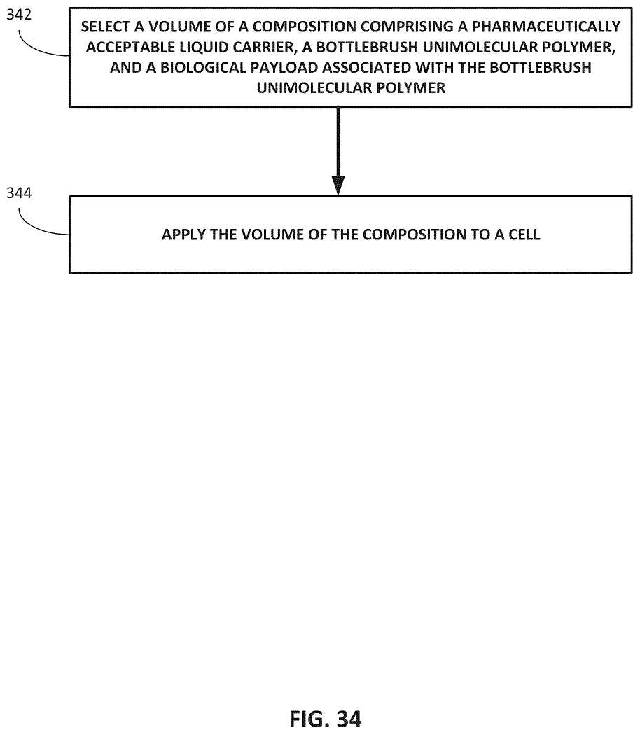

FIG. 34 is a flowchart illustrating an example technique in accordance with one or more examples of the present disclosure. The technique of FIG. 34 is described with respect to the other figures, such as FIG. 1. The technique of FIG. 34 includes selecting a volume of a composition comprising a pharmaceutically acceptable liquid carrier, bottlebrush polymer 10, and a biological payload associated with bottlebrush polymer 10 (342). The example bottlebrush polymer 10 includes backbone 12 and side-chains 14 covalently bonded to backbone 12. Each respective side-chain 14A, 14B, 14C of side-chains 14 comprises a repeating cationic unit (e.g. a monomer). The biological payload may include pDNA, or RNP, or mixtures or combinations thereof. The technique of FIG. 34 also includes applying the volume of the composition to a cell (344).

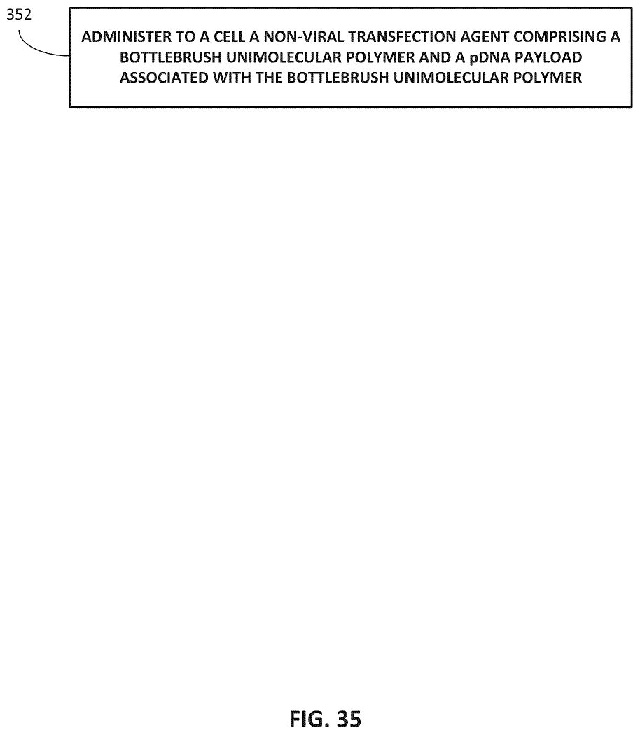

FIG. 35 is a flowchart illustrating an example technique in accordance with one or more examples of the present disclosure. The technique of FIG. 35 is described with respect to the other figures, such as FIG. 1. The technique of FIG. 35 includes administering, to a cell, a non-viral transfection agent which includes bottlebrush polymer and a biological payload associated with bottlebrush polymer 10 (352). Bottlebrush polymer 10 includes backbone 12 and side-chains 14 covalently bonded to backbone 12. Each respective side-chain 14A, 14B, 14C of side-chains 14 comprises a repeating cationic unit (e.g. a monomer). The biological payload may include pDNA, or RNP, or mixtures or combinations thereof. In some examples, administering the non-viral transfection agent may result in repair of DNA inside the cell by the biological payload.

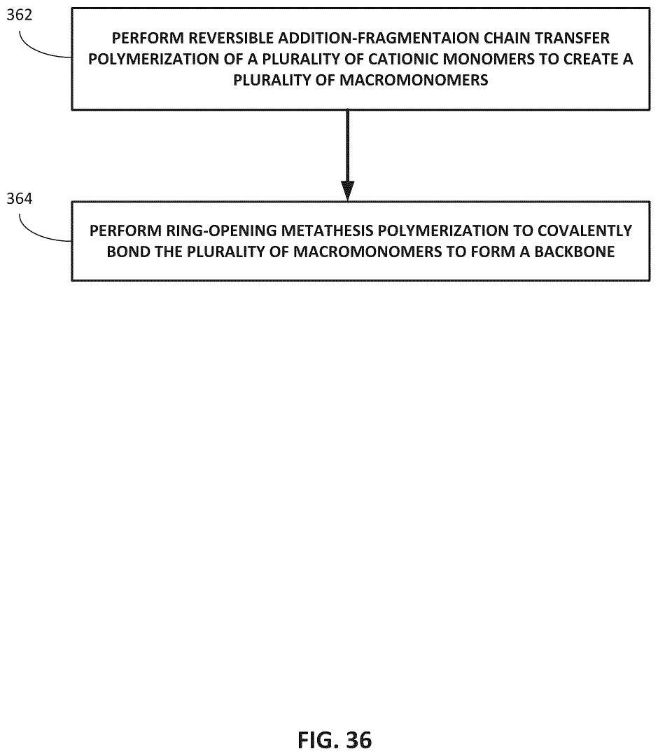

FIG. 36 is a flowchart illustrating an example technique for synthesizing a bottlebrush unimolecular polymer in accordance with one or more aspects of the present disclosure. The technique of FIG. 36 is described with respect to the other figures, such as FIG. 1. The technique of FIG. 36 includes performing reversible addition-fragmentation chain transfer polymerization of a plurality of cationic monomers to create a plurality of macromonomers 20 (362). In some examples, the cationic monomer may be 2-dimethylamino ethyl methacrylate (DMAEMA), although other monomers and co-polymers are considered. In some examples, RAFT polymerization may be performed until each macromonomer comprises from about 2 to about 1000 repeating cationic units. In some examples, performing RAFT polymerization may include reacting the plurality of cationic monomers with a chain transfer agent, which may, in some examples, be norbornene-functionalized.

The technique of FIG. 36 also includes further includes performing ring-opening metathesis polymerization to covalently bond the plurality of macromonomers 20 to form backbone 12 (364). In some examples, plurality of side-chains 14 extends (e.g., radially extend substantially perpendicularly to an axial direction defined by backbone 12) from backbone 12. In some examples, ROMP polymerization may be performed until backbone 12 comprises from about 2 to about 1000 macromonomers. In some examples, ROMP may be performed in the presence of a catalyst, such as a third-generation Grubbs catalyst. In some examples, the technique of FIG. 36 may include association a biological agent with the bottlebrush unimolecular polymer.

The following clauses illustrate example subject matter described herein:
Clause 1: A compound comprising: a bottlebrush unimolecular polymer comprising: a backbone; and a plurality of side-chains covalently bonded to the backbone, wherein each side-chain of the plurality of side-chains comprises a plurality of repeating cationic units.
Clause 2. The compound of clause 1, wherein the repeating cationic unit is 2-dimethylamino ethyl methacrylate (DMAEMA).
Clause 3. The compound of any of clauses 1 or 2, wherein each side-chain of the plurality of side-chains comprises from 2 to 1000 of the repeating cationic units.
Clause 4. The compound of any of clauses 1-3, wherein the number of side-chains in the plurality of side-chains comprises from 2 to 1000 side-chains covalently bonded to the backbone.
Clause 5. The compound of any of clauses 1-4, wherein each side-chain of the plurality of side-chains comprises a substantially equivalent number of repeating cationic units.
Clause 6. The compound of any of clauses 1-4, wherein the backbone defines a first end and a second end, and wherein the bottlebrush unimolecular polymer defines: a first side-chain covalently bonded to the first end of the backbone, a second side-chain covalently bonded to the second end of the backbone, and wherein the first side-chain comprises a greater number of the repeating cationic units than the second side-chain.
Clause 7. The compound of any of clauses 1-4, wherein the backbone defines a first end and a second end, and wherein the bottlebrush unimolecular polymer defines: a first side-chain covalently bonded to the first end of the backbone, a second side-chain covalently bonded to the second end of the backbone, a third side chain covalently bonded to the backbone between the first end and the second end, wherein the third side-chain comprises a greater number of repeating cationic units than the first side-chain and the second side-chain.
Clause 8. The compound of any of clauses 1-7, wherein the copolymer has a molecular weight, Mw of about 10 kilodaltons (kDa) to about 1000 kDa.
Clause 9. The compound of any of clauses 1-8, wherein the compound has a pKa of about 6.0 to about 9.0.
Clause 10. The compound of any of clauses 1-9, wherein the compound has a zeta potential of about 25 millivolts (mV) to about 40 mV.
Clause 11. A compound comprising: a bottlebrush unimolecular polymer comprising: a backbone; and a plurality of side-chains covalently bonded to the backbone, wherein each side-chain of the plurality of side-chains comprises a repeating cationic unit; and a biological agent associated with the bottlebrush unimolecular polymer.
Clause 12. The compound of clause 11, wherein the repeating cationic unit is 2-dimethylamino ethyl methacrylate (DMAEMA).
Clause 13. The compound of clause 11 or 12, wherein each side-chain of the plurality of side-chains comprises between 2 and 1000 repeating cationic units.
Clause 14. The compound of any of clauses 11-13, wherein the number of side-chains in the plurality of side-chains comprises between 2 and 1000 side-chains.
Clause 15. The compound of any of clauses 11-14, wherein each side-chain of the plurality of side-chains comprises a substantially equivalent number of repeating cationic units, such that each individual side-chain of the plurality of side-chains defines a substantially similar macromonomer.
Clause 16. The compound of any of clauses 11-14, wherein the backbone defines a first end and a second end, and wherein the bottlebrush unimolecular polymer defines: a first side-chain covalently bonded to the first end of the backbone, a second side-chain covalently bonded to the second end of the backbone, and wherein the first side-chain comprises a greater number of repeating cationic units than the second side-chain.
Clause 17. The compound of any of clauses 11-14, wherein the backbone defines a first end and a second end, and wherein the bottlebrush unimolecular polymer defines: a first side-chain covalently bonded to the first end of the backbone, a second side-chain covalently bonded to the second end of the backbone, a third side chain covalently bonded to the backbone between the first end and the second end, and wherein the third side-chain comprises a greater number of repeating cationic units than the first side-chain and the second side-chain.
Clause 18. The compound of any of clauses 11-17, wherein the copolymer has a molecular weight of about 10 kilodaltons (kDa) to about 1000 kDa.
Clause 19. The compound of any of clauses 11-18, wherein the compound has a pKa of about 6.0 to about 9.0.
Clause 20. The compound of any of clauses 11-19, wherein the compound has a zeta potential of about 25 millivolts (mV) to about 40 mV.
Clause 21. The compound of any of clauses 11-20, wherein the biological agent is electrostatically bound to the compound.
Clause 22. The compound of any of clauses 11-21, wherein the biological agent is at least one of a pDNA, an RNP, or mixtures and combinations thereof.
Clause 23. A composition comprising a pharmaceutically acceptable aqueous liquid carrier and the compound of clause 11.
Clause 24. A method, comprising: synthesizing a bottlebrush unimolecular polymer comprising: a backbone; and a plurality of side-chains covalently bonded to the backbone, wherein each side-chain of the plurality of side-chains comprises a repeating cationic unit; and associating the bottlebrush unimolecular polymer with a biomacromolecule to form a bottleplex.
Clause 25. A method, comprising: selecting a volume of a composition, the composition comprising: an aqueous pharmaceutically acceptable liquid carrier; and bottlebrush unimolecular polymer comprising: a backbone; and a plurality of side-chains covalently bonded to the backbone, wherein each side-chain of the plurality of side-chains comprises a repeating cationic unit; and a biological payload associated with the bottlebrush unimolecular polymer, wherein the biological payload is chosen from pDNA, RNP, and mixtures and combinations thereof; and applying the volume of the composition to a cell.
Clause 26. A method, comprising: administering to a cell a non-viral transfection agent comprising: bottlebrush unimolecular polymer comprising: a backbone; and a plurality of side-chains covalently bonded to the backbone, wherein each side-chain of the plurality of side-chains comprises a repeating cationic unit; and a pDNA payload associated with the bottlebrush unimolecular polymer, wherein the pDNA is delivered into the cell to repair the DNA of the cell.
Clause 27. A non-viral bottleplex comprising a bottlebrush unimolecular polymer and a biological agent associated with the bottlebrush polymer, wherein the bottlebrush polymer comprises: a backbone; and a plurality of side-chains covalently bonded to the backbone, wherein each side-chain of the plurality of side-chains comprises a repeating cationic unit; and wherein the biological agent associated with the bottlebrush unimolecular polymer is chosen from pDNA, RNP, and mixtures and combinations thereof.
Clause 28. The bottleplex of clause 27, wherein the bottleplex is in an aqueous solution.
Clause 29. The bottleplex of clause 27 or 28, wherein the aqueous solution is a pharmaceutically acceptable liquid carrier.
Clause 30. A method, comprising: synthesizing a bottlebrush unimolecular polymer, wherein synthesizing a bottlebrush unimolecular polymer comprises: performing reversible addition-fragmentation chain transfer polymerization of a plurality of cationic monomers to create a plurality of macromonomers; performing ring-opening metathesis polymerization to covalently bond the plurality of macromonomers to form a backbone.
Clause 31. The method of clause 30, wherein the cationic monomer is 2-dimethylamino ethyl methacrylate (DMAEMA).
Clause 32. The method of clause 30 or 31, wherein performing reversible addition-fragmentation chain transfer polymerization comprises performing reversible addition-fragmentation chain transfer polymerization until the plurality of macromonomers comprises between 2 and 1000 repeating cationic units.
Clause 33. The method of any of clauses 30-32, wherein performing ring-opening metathesis polymerization comprises performing ring-opening metathesis polymerization until the number of the plurality of macromonomers covalently bonded to form the backbone comprises between 2 and 1000 macromonomers.
Clause 34. The method of any of clauses 30-33, wherein performing reversible addition-fragmentation chain transfer polymerization further comprises reacting the plurality of cationic monomers with a chain transfer agent.
Clause 35. The method of clause 34, wherein the chain transfer agent is norbornene-functionalized.
Clause 36. The method of clause 30, wherein ring-opening metathesis polymerization occurs in the presence of a catalyst.
Clause 37. The method of clause 36, wherein the catalyst is an Ru-based catalyst.
Clause 38. The method of clause 37, wherein the catalyst is a third-generation Grubbs catalyst.
Clause 39. The method of any of clauses 30-38, further comprising: associating a biological agent with the bottlebrush unimolecular polymer.

Various aspects of the disclosure have been described. These and other aspects are within the scope of the following claims.

What is claimed is:

1. A compound comprising:
a bottlebrush unimolecular polymer comprising:
a backbone; and
a plurality of side-chains covalently bonded to the backbone, wherein each side-chain of the plurality of side-chains comprises a plurality of repeating cationic units.

2. The compound of claim 1, wherein the repeating cationic unit is 2-dimethylamino ethyl methacrylate (DMAEMA).

3. The compound of claim 1, wherein each side-chain of the plurality of side-chains comprises from 2 to 1000 of the repeating cationic units.

4. The compound of claim 1, wherein the number of side-chains in the plurality of side-chains comprises from 2 to 1000 side-chains covalently bonded to the backbone.

5. The compound of claim 1, wherein each side-chain of the plurality of side-chains comprises a substantially equivalent number of repeating cationic units.

6. The compound of claim 1, wherein the backbone defines a first end and a second end, and wherein the bottlebrush unimolecular polymer defines:
a first side-chain covalently bonded to the first end of the backbone,
a second side-chain covalently bonded to the second end of the backbone, and
wherein the first side-chain comprises a greater number of the repeating cationic units than the second side-chain.

7. The compound of claim 1, wherein the backbone defines a first end and a second end, and wherein the bottlebrush unimolecular polymer defines:
a first side-chain covalently bonded to the first end of the backbone,
a second side-chain covalently bonded to the second end of the backbone,
a third side chain covalently bonded to the backbone between the first end and the second end,
wherein the third side-chain comprises a greater number of repeating cationic units than the first side-chain and the second side-chain.

8. The compound of claim 1, wherein the bottlebrush unimolecular polymer has a molecular weight, M_{w} of about 10 kilodaltons (kDa) to about 1000 kDa.

9. The compound of claim 1, wherein the bottlebrush unimolecular polymer has a pKa of about 6.0 to about 9.0.

10. The compound of claim 1, wherein the compound has a zeta potential of about 25 millivolts (mV) to about 40 mV.

11. A compound comprising:
a bottlebrush unimolecular polymer comprising:
a backbone; and
a plurality of side-chains covalently bonded to the backbone, wherein each side-chain of the plurality of side-chains comprises a repeating cationic unit; and
a biological agent associated with the bottlebrush unimolecular polymer.

12. The compound of claim 11, wherein the repeating cationic unit is 2-dimethylamino ethyl methacrylate (DMAEMA).

13. The compound of claim 11, wherein each side-chain of the plurality of side-chains comprises between 2 and 1000 repeating cationic units.

14. The compound of claim 11, wherein each side-chain of the plurality of side-chains comprises a substantially equivalent number of repeating cationic units, such that each individual side-chain of the plurality of side-chains defines a substantially similar macromonomer.

15. The compound of claim 11, wherein the backbone defines a first end and a second end, and wherein the bottlebrush unimolecular polymer defines:
a first side-chain covalently bonded to the first end of the backbone,
a second side-chain covalently bonded to the second end of the backbone, and
wherein the first side-chain comprises a greater number of repeating cationic units than the second side-chain.

16. The compound of claim 11, wherein the backbone defines a first end and a second end, and wherein the bottlebrush unimolecular polymer defines:
a first side-chain covalently bonded to the first end of the backbone,
a second side-chain covalently bonded to the second end of the backbone,
a third side chain covalently bonded to the backbone between the first end and the second end, and
wherein the third side-chain comprises a greater number of repeating cationic units than the first side-chain and the second side-chain.

17. The compound of claim 11, wherein the biological agent is electrostatically bound to the compound.

18. The compound of any claim 11, wherein the biological agent comprises at least one of a pDNA, an RNP, or mixtures and combinations thereof.

19. A composition comprising a pharmaceutically acceptable aqueous liquid carrier and the compound of claim 11.

20. A method, comprising:
synthesizing a bottlebrush unimolecular polymer comprising:
a backbone; and
a plurality of side-chains covalently bonded to the backbone, wherein each side-chain of the plurality of side-chains comprises a repeating cationic unit; and
associating the bottlebrush unimolecular polymer with a biomacromolecule to form a bottleplex.
